(19) United States
(12) Patent Application Publication
(10) Pub. No.: US 2008/0228104 A1
(43) Pub. Date: Sep. 18, 2008

(54) Energy Assisted Medical Devices, Systems and Methods

(76) Inventors: Arthur E. Uber, Pittsburgh, PA (US); David M. Griffiths, Pittsburgh, PA (US); Alan D. Hirschman, Glenshaw, PA (US)

Correspondence Address: GREGORY L BRADLEY;MEDRAD INC, ONE MEDRAD DRIVE, INDIANOLA, PA, 15051, US

(21) Appl. No.: 10/599,366

(22) PCT Filed: Mar. 11, 2005
(86) PCT No.: PCT/US05/07829

§ 371 Date: Sep. 26, 2006

Related U.S. Application Data
(60) Provisional application No. 60/552,660, filed on Mar. 11, 2004.

(51) Int. Cl. A61H 1/00; A61B 10/02; A61M 25/06; A61B 5/15

(52) U.S. Cl. 600/567; 600/576; 601/46; 606/108

(57) ABSTRACT

A device for penetrating tissue and removing a biological sample includes a biological sampling element to remove a biological sample. The biological sampling element includes a passage therethrough. The device further includes a penetrator positioned within the passage. The penetrator is energized in a repetitive manner to assist in penetrating tissue. The biological sample element can be adapted to remove a tissue sample or a biological fluid sample (for example, blood). A device for penetrating tissue and positioning a tissue resident conduit (for example, a catheter), includes a tissue resident conduit (for example, a catheter) including a passage therethrough; and a penetrator in operative connection with the catheter. A device for inserting a tissue resident conduit includes at least one component that is energized during penetration to assist in penetrating tissue. In one embodiment, the tissue resident conduit is flexible and the energized component is positioned or a forward end of the tissue resident conduit. The device can further include a mechanism for directing the penetration of the tissue resident conduit. A needle for penetrating tissue includes a first effector including a surface and at least one actuator in operative connection with the first effector. The actuator is adapted to cause motion of the first effector such that tearing of tissue takes place in regions where there is close proximity of tissue to the surface of the first effector.

### CROSS-REFERENCE TO RELATED APPLICATION

This application claims benefit of U.S. Provisional Patent Application Ser. No. 60/552,660, filed Mar. 11, 2004, the disclosure of which is incorporated herein by reference.

### BACKGROUND OF THE INVENTION

The present invention relates generally to energy assisted devices, systems and methods, and particularly, to energy assisted medical needles, to medical needles systems and to methods of inserting needles into tissue with the assistance of energy.

A biopsy is a medical procedure that retrieves a piece of tissue from a patient for examination by a pathologist to make or to confirm a diagnosis with a high degree of certainty. The degree of certainty in the diagnosis is dependent upon obtaining a sample of the suspect tissue that is of sufficient quality for the diagnosis to be made.

There are three types of biopsies including, surgical biopsies, endoscopic biopsies, and needle biopsies. As it is desirable to cause the patient as little pain and hardship as possible, there is a trend toward biopsies using a needle rather than a knife, toward needle biopsies using finer needles, and toward image-guided needle biopsies (to make sure that the desired tissue is biopsied). Image-guided biopsy is still in its infancy, but is growing quickly.

Imaging-guided biopsies are obtained through specially designed biopsy needles that are placed into the area of concern. Needle biopsies conducted with the assistance of imaging guidance are less invasive than a traditional surgical biopsy. Many diseases, including cancer, can be detected with blood tests or seen with X-rays, computed tomography (CT) scans, magnetic resonance (MR) and other imaging techniques. When cancer is suspected, it is necessary to obtain a sample of the abnormal tissue to confirm or rule out a diagnosis of cancer. The removal of sample tissue is called a biopsy. By examining the biopsy sample, pathologists and other experts can determine what kind of cancer is present and whether it is likely to be fast or slow growing. This information is important in deciding the best type of treatment. Traditionally, biopsy has required open surgery that requires longer recovery time and typically involves the complications of pain and scarring. With interventional radiology techniques, however, tissue samples usually can be obtained without the need for open surgery.

In a large-core needle biopsy, a special needle is used that enables the radiologist to obtain a larger biopsy sample. This technique is often used to obtain tissue samples from lumps or other abnormalities in the breast that are detected by physical examination or on mammograms or other imaging scans. Because approximately 80 percent of all breast abnormalities are found to be non-cancerous, this technique is often preferred by women and their physicians. Breast biopsy procedure volumes are expected to increase over the next few years, likely a result of the increased convenience of noninvasive procedures.

Often biopsy procedures are uneventful. Sometime, especially with cancerous nodules, biopsy has been compared to trying to stick a cheap plastic fork into a grape in an opaque gel. In that regard, the mass tends to move out of the way unless the needle is directly on target, and the needle tends to bend if there is any attempt to adjust the path to the side. This bending is then exaggerated upon further forward motion because the cutting action of the needle is dependent upon the forward force applied. To resist the tendency to bow or buckle, needle diameter and/or wall thickness must be increased. It is normal practice for a doctor to lightly twist the needle by hand as they insert it. In robotic biopsy procedures, the needle is inserted at a steady pace by a machine. During such steady insertion, a patient is sometimes observed to jump or rebound when the needle penetrates a particularly tough layer of tissue. This rebound or over penetration is a significant limitation to current robotic needle biopsy processes. A similar problem occurs when a doctor tries to insert a trocar into the abdomen. There is a risk of over penetration and damage of internal organs given the force that the doctor must exert on the trocar for it to penetrate the tough abdominal wall. There are ultrasonic trocars that attempt to resolve this dilemma. The ultrasonic energy is sufficiently intense that it disrupts the cell and tissue structure, with or without sufficient heat to cauterize the hole. They are relatively large and are designed for laparoscopic or endoscopic procedures, where larger access holes are needed.

When inserting a current thin needle with beveled tip, the bevel itself causes a bending force on needle. This is because the cutting force depends upon the axial applied force. This can lead to a needle not following a straight path through the tissue. Doctors talk about using this effect as a crude form of steering. And solid and usually thicker trocar points are used if a straight path is essential

A significant biopsy risk in the abdomen is hemorrhage as a result of cutting a significant blood vessel as the needle is inserted. Bleeding complications occur most often with liver biopsy, especially when the lesion is superficial and not covered by normal liver tissue. Other complications, such as infection, are very uncommon despite the fact that the needle will occasionally traverse the bowel. In a chest biopsy, pneumothorax (air in the space between the lung and the rib cage) is the most common complication, occurring in about 25% of patients. In addition, there are a number of lesions near the rib cage that cannot be accessed with straight biopsy needles. A few fatalities from lung biopsy have occurred from puncturing an adjacent pulmonary vein. In many parts of the body, there is a risk of severing nerves. In the facial area this can lead to permanent paralysis and disfigurement.

Biopsying hard tissue or through hard tissue (to, for example, biopsy bone or the bone marrow) is especially difficult because of the stiffness of hard tissue. Bone biopsy needles must be especially strong, and thus typically have thicker walls than biopsy needles used with soft tissue and larger diameters than biopsy needle for use with soft tissue. Bone biopsy needles also typically have large T-shaped handles to exert considerable forward force upon the needle.

Spring actuated biopsy devices attempt to get around this problem by having rapid spring actuated forward motion, so rapid that the hard tissue cannot move. Side cutting spring loaded biopsy needles like the Quick-Core made by Cook, Inc of Bloomington, Ind. have the drawback that a solid needle moves through the target tissue and out the other side, possibly displacing or seeding tumor cells into adjacent healthy tissue.

The challenges discussed above in relation to biopsy also occur with needle aspiration or drainage procedures. Aspiration and drainage techniques are used to collect or remove tissue or fluid from the targeted anatomy. Similar to a biopsy, a fine needle aspiration can be used to withdraw cells from a suspected cancer. It also can diagnose fluids that have collected in the body. Sometimes, these fluid collections also may be drained through a catheter, such as when pockets of infection are diagnosed.

Needles are also used in procedures other than biopsies and aspirations. For example, needles are used to gain access to a patient's vein for the infusion of fluids or drugs. The difficulty in gaining access to a patient's vein include piercing the tough vein wall, with the vein having the tendency to move from side to side, and potentially piercing through the back side of the vein given the jerk or momentum created by the high force required for initial penetration.

Needles are also used to administer drugs subcutaneously. Especially for conditions that require multiple injections over time, such as diabetes, the smaller the needle, the less the damage to tissue and the less the pain. Also, diabetics use needles to cut the skin so a blood sample can be taken. Again, a smaller cut with the option of withdrawing blood through the needle could be beneficial.

Needles can also be inserted into the liver or other internal organs for the delivery of chemo therapy or chemo ablation. Needle electrodes are also commonly used for RF or cryo tissue ablation.

Moreover, needles are inserted into tissue to measure electrical signals from the tissue. Needles with sensors can likewise be used to measure other properties of tissue, for example, temperature, pressure, elastic properties, electrical conductivity, dielectric properties or optical properties.

Abscess drainage procedures involve the placement of drainage catheters into an abscess, guided by imaging techniques. The abscess is drained to prevent advanced infection of the localized tissue and organs. Biliary drainage procedures are generally used to relieve an obstruction to the biliary ductal system of the liver by placing a drainage catheter or stent through the patient's side and into the liver. Nephrostomy placement is the positioning of a catheter into the patient's kidney from the back. This is usually done to relieve an obstruction to the flow of urine from a tumor or some other source. A nephrostomy can be placed to allow access for removal of kidney stones, laser therapy of urothelial tumors, and the removal/dilation/stenting of strictures.

Gastrostomy placement involves the positioning of a feeding tube directly through the abdominal wall and into the stomach under x-ray guidance. It shares some of the difficulties discussed above including bleeding and difficulty cutting through tissue fascia. It is generally done for patients who will need long-term nutritional support and are not capable of maintaining their own nutritional needs orally, often for reasons such as neurological impairment, mental disorders, or severe esophageal disease including carcinoma. Gastrostomy tubes may be placed surgically, endoscopically or percutaneously.

Needles are used to suture tissue together to close a wound and promote healing. Circular solid needles are commonly used, and manipulated by the doctor using forceps or tweezers. Pushing the needle through the tissue is difficult. Even with local anesthetics, patients feel the pull and are uncomfortable or concerned. Also, the needles must be sufficiently thick/strong not to bend and to transmit the force to the tip. This increases the difficulty of moving through the tissue and trauma to the patient. Staples are a type of “needle” that are left in place for wound closure. They likewise need to penetrate tough tissue and hold the tissue together. A staple gun is often used that inserts the staple in an abrupt manner.

Needles are also used to make fluid connections, for example to penetrate rubber stoppers, for removal of a drug from or insertion of a drug into a container. Needles are also used to make fluid path connections. One of the challenges in these uses of needles is to avoid coring, that is cutting a plug from the rubber stopper or other material that then lodges in the open lumen of the needle or moves in the fluid with the risk of being injected into the patient.

In all the uses describe above, accidental needle stick injuries are a serious hazard for health care workers and patients. There are many devices for rendering a sharp needle safer by covering the tip in one of many ways. Most require some action on the part of the health care worker to activate the protection mechanism. Often this action is forgotten or improperly executed, resulting in increased risk of injury.

In the field of biopsy needles, single shot spring-loaded biopsy devices have been developed in an attempt to overcome or reduce the effect of a few of the challenges set forth above. Spring-loaded biopsy needles are inserted manually to the target tissue, and the actual biopsy is taken by actuation of a single-shot spring mechanism. There are a number of devices employing this principle on the market.

In a number of medical instruments, energy other than manual energy has been applied to effect tissue cutting, emulsification, cauterization etc. For example, an energy (that is, ultrasonic energy) assisted surgery devices exist such as the ULTRASONIC HARMONIC SCALPEL® available from Ethicon Endo-Surgery, Inc. of Cincinnati, Ohio. The energy assisted scalpel uses various levels of ultrasonic energy to cut and/or coagulate tissue, primarily during endoscopic procedure.

U.S. Pat. No. 6,514,267 also discloses an ultrasonic scalpel. It is indicated that the ultrasonic scalpel appears to transmit the ultrasonic energy more rapidly to the tissue if the scalper is relatively blunt, rather than ultrasharp. Another ultrasonic scalpel is disclosed in U.S. Pat. No. 6,379,371.

Ultrasonic energy has also been used in an instrument use to “liquefy” the lens of the eye for removal during cataract surgery. An example of such a device is disclosed in U.S. Pat. Nos. 6,352,519, 6,361,520 and 4,908,045. Although energy other than manual energy (such as ultrasonic energy) has been applied to various medical instruments as discussed above, there has little progress in developing an energy assisted medical needle. It is thus desirable to develop energy assisted medical needles, systems including such needles and methods of inserting needles using energy assistance to reduce or even eliminate some of the problems associated with the insertion of needles into tissue. Moreover, it is desirable to develop improved energy assisted medical devices generally.

### SUMMARY OF THE INVENTION

In one aspect, the present invention provides a device for penetrating tissue and removing a biological sample. The device includes a biological sampling element to remove a biological sample. The biological sampling element includes a passage therethrough. The device further includes a penetrator positioned within the passage. The penetrator is energized in a repetitive manner to assist in penetrating (that is, in entering or passing through) tissue. The biological sample element can be adapted to remove a tissue sample or a biological fluid sample (for example, blood).

As used herein in connection with effectors of the present invention, the terms “energized” or “apparatus energized” refers to the application of energy (for example, mechanical energy or thermal energy), other than by direct manual manipulation, to a penetrator (or one or more effectors thereof) of a device of the present invention such that the penetrating capability of the device is at least partially decoupled from or, in other words, not directly proportional to the forward force applied to the effector. Typically, electrical energy or stored mechanical energy is used in energizing the devices of the present invention. As used herein, the term “penetrate” refers generally to passing into or through tissue (including both soft tissue and hard tissue) through any action including, for example, cutting, tearing, cleaving, severing, ripping, emulsifying, liquefying, or ablating.

In one embodiment, the penetrator is energized continuously to assist in penetrating tissue. Alternatively, the penetrator can be energized for discrete periods of time. The penetrator can be energized in a manner to cause motion of the penetrator. In addition or alternatively, the penetrator can be energized to cause heating of the penetrator.

The motion of the penetrator can include at least one of rotational motion, lateral motion or axial motion. In several embodiments, the penetrator includes at least a single effector that is moved. The penetrator can include a plurality of effectors, at least one of which is moved. In one embodiment, the penetrator includes at least two effectors in close proximity to each other. Relative motion between the two effectors assists penetration of tissue via interaction with tissue in regions where there is close proximity of tissue to an interface between the two effectors. In another embodiment, the penetrator includes at least two effectors, a first effector which is moved and a second effector in close proximity to the first effector which is stationary. The first effector and the second effector cooperate to penetrate tissue via interaction with tissue in regions where there is close proximity of tissue to an interface between the first effector and the second effector. In a further embodiment, the penetrator includes at least two effectors, a first effector which is moved and a second effector in close proximity to the first effector which is also moved. Once again, the first effector and the second effector cooperate to penetrate tissue via interaction with tissue in regions where there is close proximity of tissue to an interface between the first effector and the second effector. As used herein with reference to effectors of the present invention, the phrases “in proximity” or “in close proximity” refer generally to a first effector, which can be moving or stationary, being close enough to a second effector, which is moving, such that the presence of the first effector affects the interaction with tissue of the movement of the second effector.

In one embodiment of the present invention, the biological sampling element includes a first tubular structure and a vibrational coupler that couples rotational energy into the first tubular structure such that the vibrational energy cuts tissue at the leading edge of the first tubular structure. The biological sampling element further includes a second tubular structure inside the first tubular structure such that the cut tissue inside the second tubular structure is protected from the effect of the rotational energy of the first tubular structure. The penetrator passes through the second tubular structure.

In another aspect the present invention provides a device for penetrating tissue and positioning a tissue resident conduit (for example, a catheter), including a tissue resident conduit including a passage therethrough; and a penetrator in operative connection with the catheter. The penetrator can include or be in operative connection with an attachment mechanism to place the tissue resident conduit in operative connection with the penetrator. The penetrator can, for example, be energized in a repetitive manner to assist in penetrating tissue. In one embodiment, the penetrator is removably positioned within the passage of the tissue resident conduit. In another embodiment, the penetrator is positioned on the exterior of the tissue resident conduit. As used herein, the term “tissue resident conduit” refers to a conduit which remains in tissue for a period of time. Typically, the period of time is in excess of 1 minute. Tissue resident conduits can also remain (typically, generally immobile) within tissue for period of time in excess of several minutes (for example, in excess of five minutes), an hour or a day. Tissue resident conduit can be flexible and include non-penetrating, non-sharp or blunted edges so that the tissue resident conduit does not penetrate, cut or otherwise damage tissue when resident therein (under generally normal use). However, in certain embodiment of the present invention energizing a tissue resident conduit can cause it to penetrate. However, once the energy is removed, the tissue resident conduit becomes generally non-penetrating. As used herein, the terms “catheter” or “cannula” refers generally to a tubular medical device for insertion into canals, vessels, passageways, or body cavities to, for example, permit injection or withdrawal of fluids or to keep a passage open. Catheters are generally flexible.

In another aspect, the present invention provides a device for inserting a tissue resident conduit including at least one component that is energized during penetration to assist in penetrating tissue. In one embodiment, the tissue resident conduit is flexible and the energized component is positioned or a forward end of the tissue resident conduit. The device can further include a mechanism for directing the penetration of the tissue resident conduit.

In another embodiment, the device includes a rigid penetrator and the energized component is positioned on a forward end of the penetrator. The tissue resident conduit is in operative and removable connection with the penetrator so that the penetrator can be removed from the penetrated tissue while the tissue resident conduit remains within the penetrated tissue. In one embodiment, the penetrator includes an axial passage therethrough in which the tissue resident conduit is positioned during penetration. In another embodiment, the penetrator is positioned within the conduit during penetration. In still another embodiment, the tissue resident conduit is positioned adjacent the penetrator during penetration. The penetrator can, for example, be adapted to penetrate through the wall of a blood vessel.

In one embodiment, the tissue resident conduit is flexible. The tissue resident conduit can, for example, be a catheter.

In another aspect, the present invention provides a needle for penetrating tissue including a first effector including a surface and at least one actuator in operative connection with the first effector. The actuator is adapted to cause motion of the first effector such that tearing of tissue takes place in regions where there is close proximity of tissue to the surface of the first effector. In general, as used herein, the term “tear” refers to separating parts of the tissue or pulling apart the tissue by force. In general, “cutting” refers to penetration with an edged tool or to a dividing into parts with an edged tool.

In one embodiment, the surface of the first effector is a forward surface thereof. The forward surface of the first effector can be rough or abrasive. In general, a rough surface is marked by inequalities, ridges, or projections on the surface. The roughness or abrasiveness assists in “gripping” of tissue contacted by the surfaces so as to provide resistance to movement of the tissue relative to the forward surface.

In one embodiment, the needle penetrates without application of a significant axial force thereto.

The tissue can be torn along a path determined by the characteristics of the tissue. The path is generally determined at least in part by the resistance to tearing exhibited by tissue forward of the needle. Tissue having a relatively higher resistance to tearing can be pushed aside by the needle and not torn.

The needle can further include at least a second effector having a surface. The surface of the second effector is in close proximity to the surface of the first effector. Relative motion between the first effector and the second effectors causes tissue tearing to occur in regions where there is close proximity of tissue to an interface between the first effector and the second effector.

In a further aspect, the present invention provides a needle for penetrating tissue including a first effector including a surface and a second effector including a surface. The surface of the second effector is in close proximity to the surface of the first effector. The device further includes at least one actuator in operative connection with one of the first effector and the second effector. The actuator is adapted to cause relative motion between the first effector and the second effectors such that tissue penetration takes place in regions where there is close proximity of tissue to an interface between the first effector and the second effector.

In another aspect, the present invention provides a needle for sampling tissue including a first tubular structure and a vibrational coupler that couples rotational energy into the first tubular structure. The vibrational energy is suitable to penetrate tissue at the leading edge of the first tubular structure. The device further includes a second tubular structure positioned inside the first tubular structure, such that cut tissue passes into the second tubular structure and is protected from the effect of the rotational energy of the first tubular structure.

In still another aspect, the present invention provides a needle for penetrating tissue including a first effector in proximity to the distal end of the needle; and at least one actuator in operative connection with the first effector to energize the first effector to assist in penetrating tissue.

In another aspect, the present invention provides a needle system including a needle in operative connection with a syringe and an actuator in operative connection with the needle. The actuator is adapted to energize to the needle to assist in penetrating tissue. The needle can, for example, be connected to the syringe by a hub, wherein the hub allows relative motion between the needle and the syringe. The needle and the syringe can both be energized. In one embodiment, the actuator is in operative connection with a cradle in which a needle and syringe are insertable to energize the needle.

In another aspect, the present invention provides a method of inserting a needle into tissue, including the step of energizing at least a forward end of the needle to assist in penetrating tissue.

In still a further embodiment, the present invention provides method of inserting a tissue resident conduit (for example, a catheter) into tissue, including the step of energizing at least a portion of a forward end of an insertion device to assist in penetrating tissue. The tissue resident device can be flexible. The tissue resident device can also have a blunt forward surface.

In a further aspect, the present invention provides a device for penetrating tissue including a nonlinear penetrator. The nonlinear penetrator includes at a forward end thereof at least a first effector. The device further includes at least one actuator in operative connection with the first effector. The actuator is adapted to cause motion of the first effector. The penetrator can be curved with a curve of a predetermined shape. The curve can have a constant radius of curvature or a varying radius of curvature. The penetrator can be curved in a simple or a complex manner. As used herein, the term “complex” refers to a curved section that curves in more than one direction or more than one plane. In one embodiment, the penetrator is flexible. The device can further include a mechanism to direct the penetration of the penetrator.

In another aspect, the present invention provides a device for penetrating tissue including a penetrator including at a forward end thereof at least a first effector and at least one actuator in operative connection with the first effector. The actuator is adapted to cause motion of the first effector. The first effector is rotatable about the axis of the penetrator

In another embodiment, the present invention provides a non-coring needle including a penetrating member. A forward end of the penetrating member includes a forward extending section including at least two points spaced from each other and being adapted to pierce tissue. The needle can further include an actuator to energize at least a portion of the needle to facilitate penetration. At least a portion of the forward end of the penetrating member can be non-cutting so that coring does not occur upon penetration of the tissue. In one embodiment, the at least two point are positioned to stabilize tissue for penetration. An example application of this needle is holding a blood vessel stable for puncture at an angle.

In still a further embodiment, the present invention provides a blunt needle including at least one effector that does not readily penetrate tissue and at least one actuator in operative connection with the effector that when energized enables or enhances the ability of the effector to penetrate tissue. The needle can contain a conduit such that fluid can be delivered to the tissue or material removed from the tissue.

In general, the energy assisted devices and systems of the present invention can be used in practically any medical procedure requiring penetration, hole boring or incision of tissue including, for example, biopsies of both soft and hard internal tissue; removal of tissue for therapy (for example, cataract removal); cauterization, incision (that is, surgery), needle access to veins, arteries, or other blood vessels for blood testing (including small sample blood testing as, for example, practiced by diabetics) aspiration, drainage access, gastrostonomy, chemical or RF ablation, sensor access to tissue and drug delivery to target tissue. Several advantages are provided over common instruments (including needles) currently used in such procedures. In general, these advantage are afforded by at least partially decoupling the penetrating or cutting action of the devices of the present invention from the forward force applied thereto. For example, smaller needles can be used, less push force is require, less “tug” force is felt by the patient, there is less of a tendency of deflection from the desired path, a curved path can be followed, the path can be changed during insertion, and there is less bleeding and damage to tissue. Patient pain can further be reduced with the devices of the present invention by, for example, local injection of an anesthetic, local affecting of nerves via applied electrical energy, local affecting of nerves via applied vibrational energy, air exclusion and/or the tissue penetrating profile of the device.

### BRIEF DESCRIPTION OF THE DRAWINGS

Other aspects of the invention and their advantages will be discerned from the following detailed description when read in connection with the accompanying drawings, in which:

FIG. 1 illustrates a block diagram of one embodiment of an energy assisted needle system of the present invention.

FIG. 2 is a cross-sectional illustration of one embodiment of the patient or distal end of an energy assisted needle.

FIGS. 3a, 3b, and 3c are illustrations of other embodiments of the patient or distal end of an energy assisted needle.

FIG. 4 is an illustration of a further embodiment of the patient end of an energy assisted needle using axial motion for penetration.

FIG. 5 is an illustration of the patient end of any of the energy assisted needles of FIG. 2, 3 or 4 with the center penetrating assembly removed so that a sample of tissue can be taken.

FIG. 6 is an end on or top view of the actuator end of the energy assisted needle including a mechanism to couple rotational motion to the effectors.

FIGS. 7a, 7b, and 7c are illustrations of the actuator end of the energy assisted needle including a mechanism to transform longitudinal motion into rotational motion of the effectors.

FIG. 8 is an illustration of one embodiment of an energy assisted needle system including a disposable needle.

FIGS. 9a and 9b illustrate embodiments of a tissue cut-off device.

FIGS. 10 and 10b illustrate an embodiment of an energy assisted IV catheter.

FIGS. 11a, 11b, and 11c illustrate a currently available non-coring needle tip

FIGS. 11d. 11e, and 11f illustrate a multi-point needle for improved access to vessels and tough tissue.

FIG. 12a illustrates problems accessing a site with a linear needle.

FIG. 12b illustrates an embodiment of a guide for a curved energy assisted needle.

FIGS. 13a and 13b illustrate embodiment of a curved energy assisted needle.

In the figures, each identical or nearly identical component that is illustrated in various figures is represented by a single numeral. For purposes of clarity, not every component is labeled in every figure, nor is every component of each embodiment of the invention shown where illustration is not necessary to allow those skilled in the art to understand the invention.

### DETAILED DESCRIPTION OF THE INVENTION

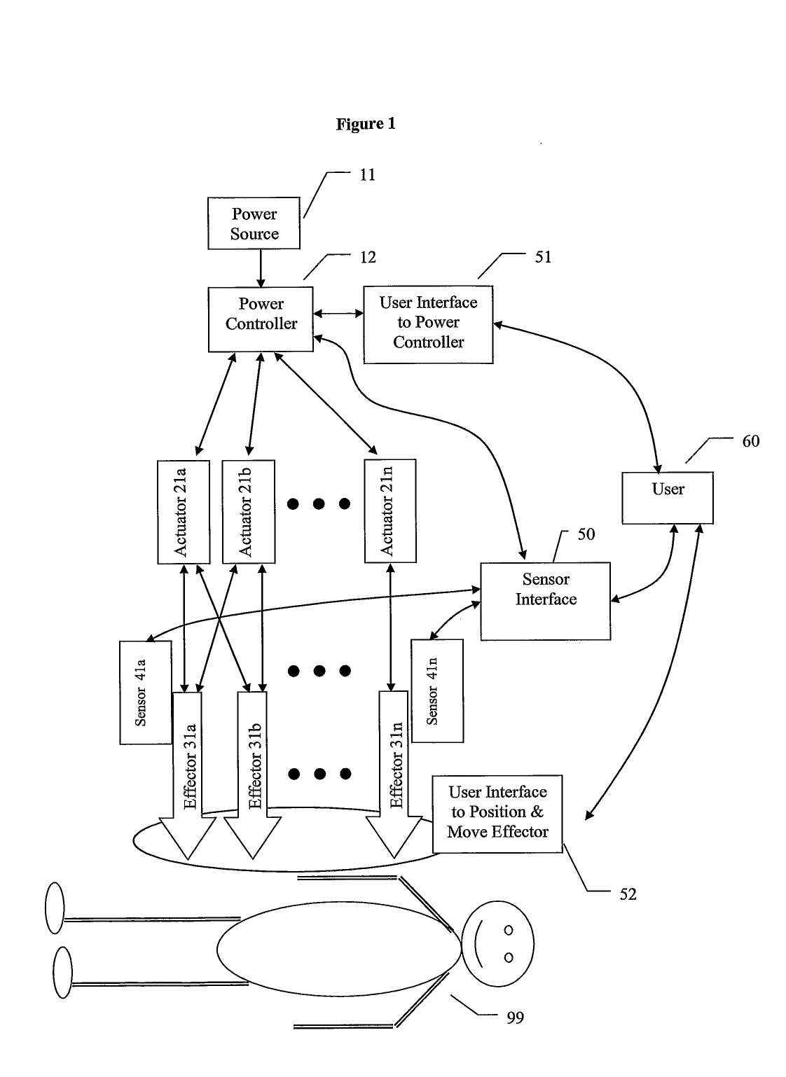

The energy assisted systems of the present invention can be used in connection with a number of medical devices and/or procedures. However, the systems of the present invention are discussed primarily herein in connection with representative embodiments of energy assisted “needles”. FIG. 1, for example, illustrates a block diagram of an energy assisted needle system of the present invention that will be used to discuss the general functionality of various embodiments of energy assisted needles of the present invention. As used herein, the term “needle” refers to relatively slender instruments that can be used to penetrate, and includes instruments having a passage or channel for introducing material into or removing material from the body parenterally. In common language, needles tend to be sharp and rigid whereas catheters are non-cutting and usually soft and flexible. With energy assistance, the distinction blurs because soft materials (such as the materials used in catheters) can cut. Therefore needles encompass as a subset both needle-catheter systems such as used for vascular access and catheters. Needles in this context can also be solid, have multiple independent or communicating passages, and be made of various materials and construction styles.

In system 10, power or energy is provided by a power source 11. A number of different types of energy can be used in the systems of the present invention. Electrical energy can be provided from batteries, fuel cells, line power, or similar devices. Mechanical energy can be provided by compressed air, hydraulics, or spring power. It can be in the form of oscillatory or steady energy or motion.

The power or energy is controlled through a power controller 11 such that one or more actuators, 21a, 21b, . . . 21n, create actions or motions. For example, mechanical actions or motion can be created from electrical power by any of many electromechanical elements, for example solenoids, motors (including, for example, linkages or cams), piezoelectric elements, ultrasound transducers, electroactive actuators (for example, shape memory alloys such as nitinol, electroactive polymers, and electroactive ceramics), magnetostrictive elements, and electrostrictive elements. Hydraulic elements and pneumatic elements can also be used to create mechanical actions. Examples of these are air or hydraulic motors or turbines and various cylinders or bellows. Pneumatic and hydraulic (using saline or water for example) has the advantage that the needle and associated actuators could be built simply, sterilized as one unit, and then be disposed of after a single use. Likewise, thermal energy can be used in the form of, for example, heat/shock from electrical elements, and lasers can create photon energy. Vacuum can be used to power actuators and to urge tissue towards one or more effectors. Motion can be created as for example in electric toothbrushes or using eccentric weights on a motor as in U.S. Pat. No. 5,299,354 and U.S. Pat. No. 5,647,851 the disclosures of which are incorporated herein by reference. A motor can be reused and mated with a disposable segment, for example as show in U.S. Pat. No. 5,324,300 included herein by reference.

These actuators 21a, 22b . . . 21n act upon one or more effectors 31a, 3b . . . 31n which transmit the effect, the energy, to the patient 99, achieving the medical goal of the user 60. Effectors 31a, 31b . . . 31n are preferably associated with each other or held together by an interface 52 which can be used to position and move effectors 31a, 31b . . . 31n. In FIG. 1, interface 52 is shown diagrammatically as a box and an oval encompassing effectors 31a, 31b . . . 31n.

User interface 52 can for example be a hand-held interface. Alternatively, user interface 52 can be part of a robotic or automated interface. The control of interface 52 can be partially or fully automated. As described below, feedback can be provided to user interface 52 to assist in control thereof. Guidance of user interface 52 can be manual, machine assisted, or fully machine controlled (such as robotic biopsy). 3D position monitors can, for example, be positioned on the patient and/or on one or more effectors and/or on effector user interface 52. As known in the art, various imaging systems can be used to facilitate guidance of interface 52 (and thereby effectors 31a, 31b . . . 31n. For example, ultrasound imaging, X-ray imaging, CT imaging, and/or MR imaging, microscopes, endoscopes or laparoscopes can be used in connection with either manual or machine assisted guidance. There are a number of systems that provide some type of feedback for guidance. For example, an image of the needle tip, the anticipated path if motion continues as aimed, and the target tissue can be provided so that the doctor can make sure the needle is heading to the right tissue, is avoiding any tissue that could be damaged, and samples the target tissue with confidence. This is generally termed 3D guidance. Ultrasound transducers with attached disposable or reusable needle guides are a common device used to provide real time visualization of the needle and the target as a needle is being inserted. Various other systems use images to calculate a needle path and then have a mechanism such as angle guides or laser guides to help make sure the doctor places the needle at the proper angle and goes to the correct depth. Stereotactic head frames are an example of this assisted introduction. In the MAMMATOME® Breast Biopsy System available from Ethicon Endo-Surgery, a coordinate system directs the biopsy needle to the proper location. Tremor cancellation devices are being built to assist with surgery, for example on a beating heart. Such devices may also be applied to improve biopsy procedures.

One or more sensors 41a . . . 41n can be associated with any of the effectors 31a, 31b . . . 31n, actuators 21a, 21b . . . 21n, the patient 99, or any of the other system components. The sensors communicate with a sensor interface 50 so that information can be given to the user 60 or other equipment for monitoring, controlling, or other functions. The sensor information can also be fed to the power controller to provide feedback control. Sensors 41a . . . 41n can, for example, sense tissue properties (for example, water content, fat content or other properties). Sensors can, for example, include durometers, conductivity sensors, dielectric property sensors, optical sensors, strain gauges, ultrasound reflectance sensors and microelectromechanical-system (MEMS) sensors.

Sensors can also be used to provide, for example, audible or tactile feedback to the user. For example, sensors (such as strain gauges and/or other sensors) on effectors 31a, 31b . . . 31n can sense resistance to motion, forward motion, bending, friction and/or temperature to provide feedback to the user. This feedback can, for example, alert the user to undesired bending or path deviation. Such feedback can also indicate desired conditions, such as penetration of a vein wall or penetration into bone marrow.

The sensors may also provide diagnostic information. In some cases the sole purpose of placing the needle in the tissue may be to make a measurement via the sensor, for example temperature, pressure, or chemical.

Sensor interface 50 can communicate with the power controller, which can modulate the power applied to one or more actuators based upon the information of one or more sensors. And example of this is to provide an effect similar to power steering or power brakes which provides power assist and yet maintains relative tactile feedback to the user, such that when a sensor 41a, 41b, . . . 41n senses an increased force resisting forward motion, the power to the appropriate actuator can be increased to increase the cutting action and thus reduce the resistance to forward motion to its desired level in relation to the forward force of the operator or system. Cutting action can also be quickly changed (for example, reduced or stopped) when forward resistance increases significantly, for example coming up against the bone, or when forward resistance decreases significantly, for example penetrating a vein, bone, or the abdominal wall.

The user can directly interface through the user interface to the power controller, for example, to control cutting level or simply to turn the cutting action on when the needle is use or to turn the cutting action off when the needle is not in use, thereby making the needle inherently less of a needle stick risk. The arrows between the system blocks of FIG. 1 represent transmission of energy, information, control, or communications.

In general, motion is applied to one or more effectors 31a, 31b . . . 32n via actuators 21a, 21b . . . 21n, respectively. Many different types of motion can be applied to effectors 31a, 31b . . . 31n. Moreover, the type of motion applied to one or more different effectors can be different. In general, the motion applied is preferably repetitive. The motion can be applied continuously or for discrete periods of time. Example of types of motion applicable to effectors 31a, 31b . . . 31n include, but are not limited to rotation (for example, unidirectional, reciprocating, random or arbitrary, hammer drilling etc.), linear motion either axially or perpendicular to the needle axis (for example, oscillatory, random, impulse transmitted and hammering), arbitrary directional motion and combined motion. Combined motions can be as simple as rotational motion about the axis and reciprocal motion along the axis. Or it can be as complicated as a geological tunnel boring action where, for example, there is overall rotation and there is rotation of many cutter elements within the overall rotation. Effectors can act in coordination as in two cooperating moving surfaces. Effectors can also act in cooperation with a stationary surface. Alternately stationary surface can be considered as an effector with zero motion, for example to protect tissue from the motion or other effectors.

The gross motion(s) or path of the needle can follow a curve (including arbitrary curves and complex curves). Following a curve can, for example, be advantageous in biopsies in which obstacles (for example, ribs, major blood vessels and/or nerve bundles) are to be avoided. Normal needles cannot be curved because the cutting force has to be provided by the user at the end opposite of the cutting, and this will tend to cause them to buckle. There are curved needles that reach into open cavities, such as laryngeal needles, or needles with curved segments that are inserted through straight needles and then allowed to curve upon exiting the large, stiff straight needle. But usually these curved segments curve in opens spaces such as a chamber of the heart in the abdominal cavity where organs cam move with respect to each other, or in the lungs, brain, or bone marrow which are relatively soft. But, in all cases, curved needles are significantly thicker than would be necessary for a similar straight needle because of the bending stress that must be withstood. This increases trauma to the patient.

Needle guides or stereotactic head frames can, for example, be modified to accommodate curved needles of this invention. 3D guidance devices can likewise show the path that the curved needle would follow. Curved needles can, for example, be provided with discrete standard curvature radii so that guiding devices and needle path software can be adjusted to accommodate the needles. Curved needle guides adhesively attached to the skin can also be used.

Curved needles of the present invention can, for example, be simple curves or curved in multiple directions and/or planes (for example, spirals). Techniques from steerable laparoscopes, endoscopes, or robotics can, for example, be applied to allow an arbitrary access path to be achieved to a target because the cutting action at the tip is independent of the forward thrust or force.

In general, motions applied to effectors 31a, 31b . . . 31n of the present invention can vary in rate, frequency, amplitude and duration/timing of application. The frequency of oscillatory motions can vary over a wide range. For example, the frequency can be less than 1 Hz. Likewise, the frequency can be in the range of approximately 1 to 10 Hz. The frequency further can be in the range of approximately 10 to 1000 Hz, in the range or approximately 1 kHz to 10 kHz, in the range of 20 kHz to 2 MHz or greater than 2 MHz. At higher frequencies, the amplitude of the motion is limited as a result of the acceleration required to reverse the direction. In the case with combinational motion, it is preferred that the motions be of the same frequency, of harmonics of each other, of slightly different frequencies, or of significantly different frequencies. Examples will be given later

The structure of effectors 31a, 31b . . . 31n can be varied. For example, the forward surface(s) or tip(s) of effectors 31a, 31b . . . 31n can be sharp or pointed (including, for example, a single or multiple bevels). Standard and custom needle points can, for example, found in the OEM Services brochure of Popper & sons of Lincoln, R.I. or on the web site of Connecticut Hypodermics of Yalesville, Conn. An advantage of providing an energy assist to the effectors is that the surfaces are not limited to the normal sharp designs. The surfaces can also be rounded or blunt. The surfaces can further be smooth or rough on, for example, a micron scale or a tens of micron scale. Likewise, a variety of action surfaces can be provided. For example, in the case of a single action surface, the surface can be spiral as in a corkscrew, as in U.S. Pat. No. 4,919,146. A rotating scoop-like surface can also be used. In the case of a single action surface, a second surface can be provided as an action stop or shield. In the case of action between two surfaces, the surfaces can cooperate as in a cutter and anvil, an electric knife or as in opposing “Pac Man” jaws. The two surfaces can act in a coordinated fashion or independently. Multiple thrusting elements (which are activated for example, similarly to the wires used in dot matrix printers—see, for example, U.S. Pat. No. 4,802,781, the disclosure of which is incorporated herein by reference) can be provided which operate in tandem and/or sequentially. Additionally, force can be applied through application of fluid jets or through a vacuum (wherein, for example, tissue is pulled against a surface).

The cross-sectional shape of effectors 31a, 31b . . . 31n in can vary widely. For example, the effectors can be conformed to be rotationally symmetric, to be a rectangular shape or a thin straight line, to be multiple lines initiating from a center, to be multi pointed (star patterns) or to lack symmetry. These shapes may be chosen to provide the desired cut pattern or cross section.

The effectors can be straight and rigid over the length thereof or be rigid and curved. One effector can, for example, provide the primary shape and that the other effectors can be relatively flexible and thus able to conform to the shape of the rigid effector. This is, for example, the can for an embodiment of a curved needle, in which one or more effectors are sufficiently stiff to define the shape and other effectors are flexible enough to move or be moved in relation to the shape defining effector(s). Moreover, overall or general flexibility can be provided. Preferably such flexibility can be controlled or steered by the user by, for example, methods similar to those currently used in connection with steerable laparoscopic devices or steerable catheter devices.

As certain effectors of the present invention move with respect to each other, friction between them is preferably limited. This requires sufficient tolerances to ensure clearance between adjacent effectors. Surface treatments such as Teflon or “hard coating” can be used. Surface treatments can be used to increase smoothness and thus reduce friction. Or, materials can be chosen to provide inherent lubricity, such as a smooth metal mated with high-density polyethylene or Teflon. Liquid lubricants such as silicone oil can be inserted between effectors in manufacture. A liquid for lubrication, such as physiological saline, can be injected between effectors during use.

Because the penetrating or cutting effort has generally been separated from forward force, the effector materials can be expanded beyond the traditional needle material of stainless steel or other metals. Consider a paper cut; energy in the form of relative motion allows a very weak and flimsy material to make a quick precise incision. While paper is not stable in a moist environment, thin plastics or ceramics may be used for effectors. Especially plastics loaded with abrasive particles could be beneficial if the abrasives can be selectively exposed or applied on the patient end by melting, grinding, solvents, or other means during manufacture. And, if metals are used, very thin metal effectors are advantageous.

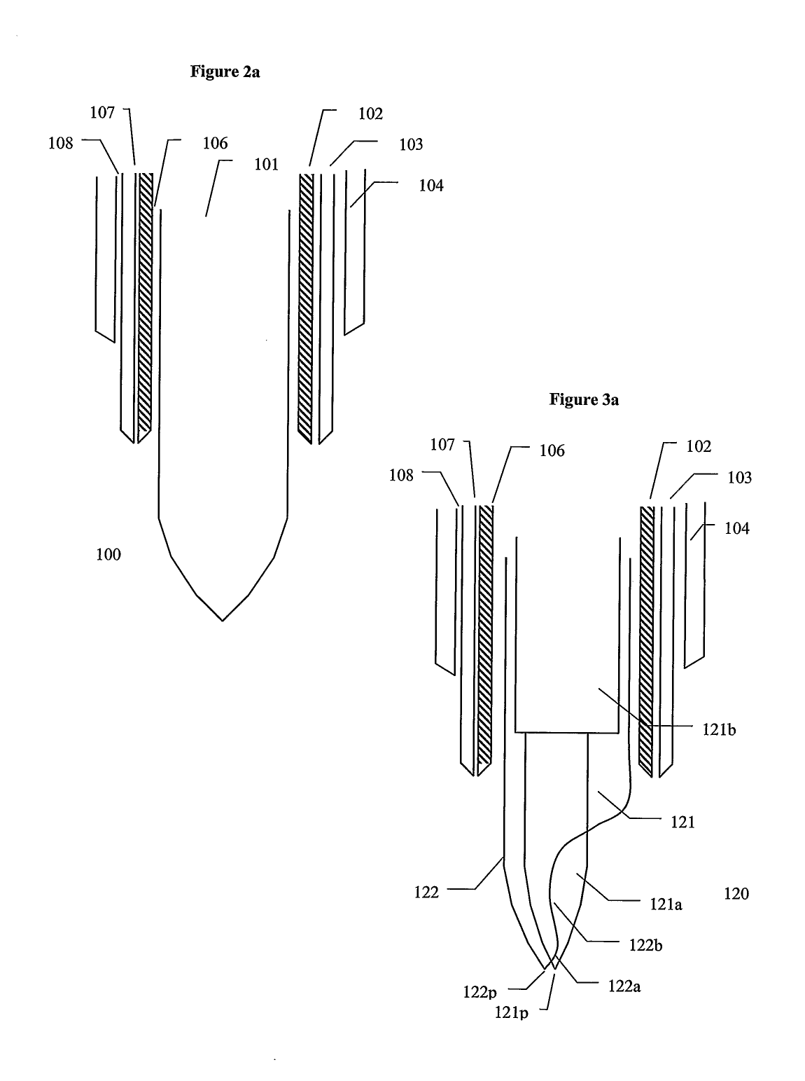

FIG. 2 illustrates one embodiment of an energy assisted needle that can be used in the system of FIG. 1. In that regard, FIG. 2 illustrates a patient end 100 of an embodiment of the energy assisted needle. The energy assisted needle includes a central core or shaft, often called a stylet 101 that is generally pointed and can have a rough or abrasive surface, for example similar to a very fine file, rasp, sandpaper, machine tool, or grating. A rough surface is one marked by inequalities, ridged, or projections on the surface that assist in gripping of tissue. Coring tubes 102 and 103 are generally concentric with core 101. Sheath 104 is generally concentric with all of these. Elements 101, 102, 103, and 104 are referred to by the general term “effectors’ because they effect (or prevent an effect on) the tissue in one way or another or at one time or another. There are four effectors in the embodiment of FIG. 2.

To penetrate tissue, stylet 101 is moved or agitated. This agitation can, for example, be unidirectional rotation at a rate that does not cause significant heating. Likewise, the agitation can be a reciprocal motion, rotationally and/or axially, similar to the operation of a jackhammer. The motion can optionally have an orbital aspect to it as well. The rough surface of stylet 101 abrades and tears the tissue so penetration is easier than without energy assistance. The tearing force and action occurs due to the motion of the effector 101 in relation to the tissue. As described above, other motions or combination of motions can be used. The areal extension of the rough surface is selected to balance the tissue penetration capability against the tissue damage done. The rough surface of stylet 101 can be randomly rough, or it could have a spiraled pattern of groves and edges that tends to separate tissue along fascia. The benefit of separating tissues along their “grain” is that the likelihood of penetrating or severing larger blood vessels or major nerves is reduced. The actual tear or separation plane or path in the tissue is defined and influenced by the characteristics of the tissue than by the edges of the effector. This is in contrast to current needles where the cutting path and surface is determined by the sharp cutting edge of the needle. The needle of the present invention is effectively following the “path of least resistance” to the target, moving higher resistance structures out of the way. This also reduces the damage, especially bleeding, and thereby increases the speed of healing. Stylet 101 could optionally have a very sharp or pointed section right at the tip (either on axis or preferably somewhat off axis) to speed penetration and only minimally increase the chance of damaging tougher tissue structures such as blood vessels. In this case, the cutting energy is focused over a tiny area, and only a very tiny cut is made and the remainder of the hole is from the action of teasing or tearing the tissue apart.

Clearance channel 106 between core 101 and effectors 102, clearance channel 107 between effectors 102 and 103, and clearance channel 108 between effectors 103 and 104 can be used to deliver or remove fluids such as saline, coolant, local anesthetics, and disinfectants to or from the cutting areas. Channels 106, 107, and 108 also provide separation or clearance between effectors 101, 102, 103, and 104, should distinct motions be desired.

With stylet 101 in place and energy applied, the needle penetrates into tissue or other material without cutting a core or a sample. Tissue is just stretched and moved out of the way. Examples of suitable actuators for this embodiment are discussed elsewhere herein.

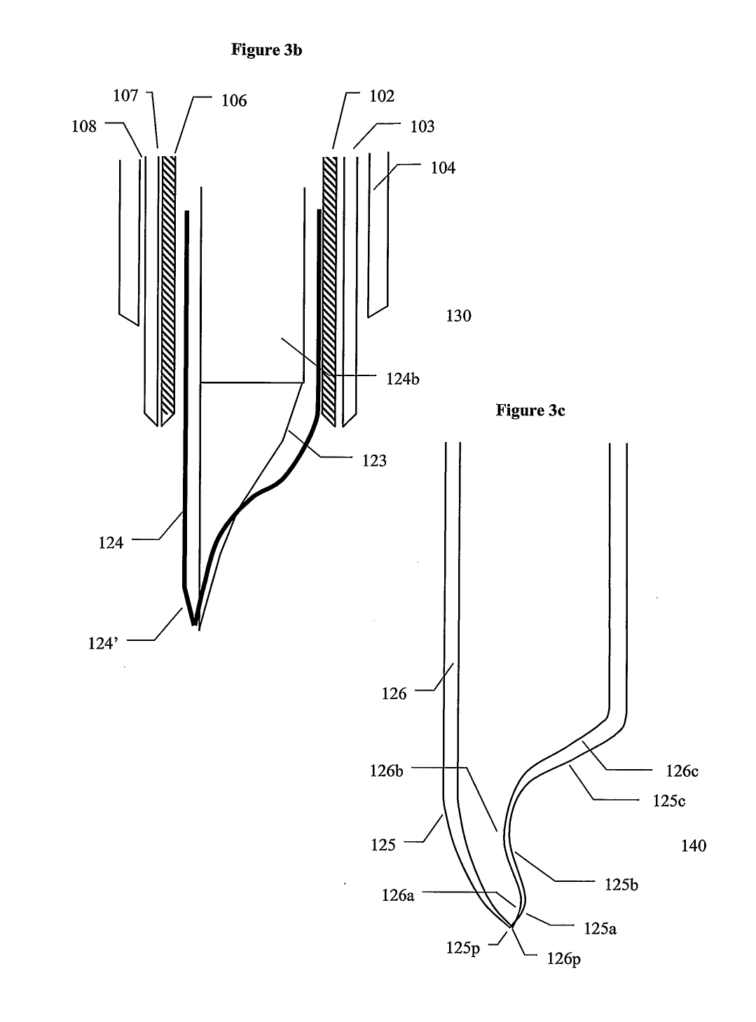

FIG. 3a shows an alternative embodiment of an energy assisted needle with two effectors 121 and 122 forming the stylet. The actuators are arranged and powered so that there is relative motion between effectors 121 and 122. For example they can both rotate, either in opposite directions or in the same direction with different speeds. Alternatively, one effector can remain still and the second effector be moved. In FIG. 3a, effector 121 is shown as having two different parts on the patient end. The axis of symmetry the tip 121 a is slightly different than the axis of rotation of the main shaft 121b. Thus, as effector 121 is rotated, the tip segment 121a moves closer to and away from the tip of effector 122. This motion can provide the benefit of “teasing” or tearing apart tissue along fascia. This teasing action reduces the tendency to cut significant blood vessels or nerve bundles. Effector 121 establishes a grip on the tissue at one point, and effector 122 establishes a grip on the tissue at a second point. The energy assisted motion then moves these two points apart, causing the tissue to tear along a line defined by tissue properties interacting with the direction and amplitude of motion of the effectors. In selected embodiments the tearing comes from motion generally perpendicular to the axis of the needle. Alternatively, relative motion generally parallel to the axis of the needle can be translated into separating force via the wedge or ramp shaped surfaces of one or more of the effectors. Alternatively, relative rotational motion around the axis of the needle can be translated into separating force via non-rotationally symmetrical surfaces on one or more of the effectors. As discussed elsewhere, this separation via tearing or teasing is in contrast to the action of scalpels, knives, scissors, saw blades and similar cutting edges, either singly or in opposition, where the tissue is severed or cut along lines primarily determined by the geometry and motion of cutting edge. Effector 121 can alternatively be appropriately sized and effector 122 can be appropriately sharpened so that there is cutting action only at the very tip, or along surface 122a, or along surfaces 122a and 122b, which effectively cuts a line through the tissue being penetrated. Examples of actuators for this embodiment are also discussed elsewhere herein. Or, selected surfaces of effectors 121 and 122 may be rough on the macroscopic or microscopic scales to promote tissue gripping and tearing.

FIG. 3b shows a second embodiment with the “teasing” mode of separating tissue. Here the teasing takes place at the edge, rather than in the middle of the needle. This can be especially useful with curved needles because the off axis tearing and/or cutting can be used to cause the needle to inherently bend in the direction of the curvature of the needle. (Doctors currently use a similar effect with manual beveled needles to provide a limited or slight amount of directional control.) TA sharp point is created by beveling effector 124 as indicated at 124′. This allows adjacent edges of 123 and 123 to move in very close proximity to each other, alternately teasing apart tissue or cutting through tissue dependent upon the details of the edge created by grinding or machining and upon the direction, amplitude, and speed of relative motion. A relative rotational motion of 10 to 20 degrees would tend to cut, similar to a miniature electric carving knife. Then the relative motion of the tapered sections of 123 and 124 would enlarge the hole in the tissue. Relative axial motion or relative side to side translational motion would tend to tear the tissue more than cut it, and so reduce even further the chance of cutting significant blood vessels.

In FIGS. 3a and 3b, effectors 102, 103, & 104 are show in cross section in a plane containing the axis of the assembly and are generally cylindrical. Effectors 121 and 123 are also shown in cross section. Effectors 122 and 124 are not a cross section but are shown as viewed from the outside, so that it is possible to better understand how the curved surfaces of the effectors interact.

FIG. 3c shows a device with two effectors, 125 and 126. The effectors are one inside the other, and the curved end and edges are constructed so that they selectively cooperate. To penetrate, relative motion, optionally mostly axial will enable points 125p and 126p to cooperate to separate the tissue and facilitate penetration. Alternatively edges 125a and 126a can be cutting edges to cut through tissue during penetration. In this penetrating mode, the energy direction and or amplitude is such that edges 125b and 126b and edges 125c and 126c do not interact. When the site for a tissue sample or biopsy is reached, the amplitude and or direction of energy is increased so that all sharp edges provide a cutting action. By moving the effectors forward in the tissue and rotating the effector assembly in the tissue in a synchronized manner, a spiral sample of tissue can be cut. With a slow enough forward motion, a solid cylinder of tissue can be sampled. To facilitate separation of the tissue sample from the patient's remaining tissue, forward motion is stopped, decreased, or reversed and a full 360 rotation of the effector takes place. If the point 125p is at or past the center axis of the device, the tissue can be severed. If the point 125p does not reach the center axis, the tissue sample will be partially severed. This weakening of the connection to the remaining tissue allows the sample to be more reliably extracted, especially with the curvature on the tip helping to hold the needle in place. If need be, a slight sideways motion could be used to sever the remaining connection. In this embodiment, the effector surface away from edges 125a, 125b, and 125c is a closed smooth surface. The opening in which tissue enters the effector is from the side of edges 125a, 1225, and 125c.

The device of FIG. 3c also has the operational advantage that it can change from non-coring penetration to tissue sampling or coring without the need to change any effector elements. This enables non-contiguous sampling along a single needle path at the tip of the needle. It also has the advantage that the tissue sample is cut at the forward part of the needle, eliminating the need to transverse the potential tumor and thereby minimize the possibility of tumor seeding into healthy tissue, and allowing automatically separated of the tissue sample from the patient.

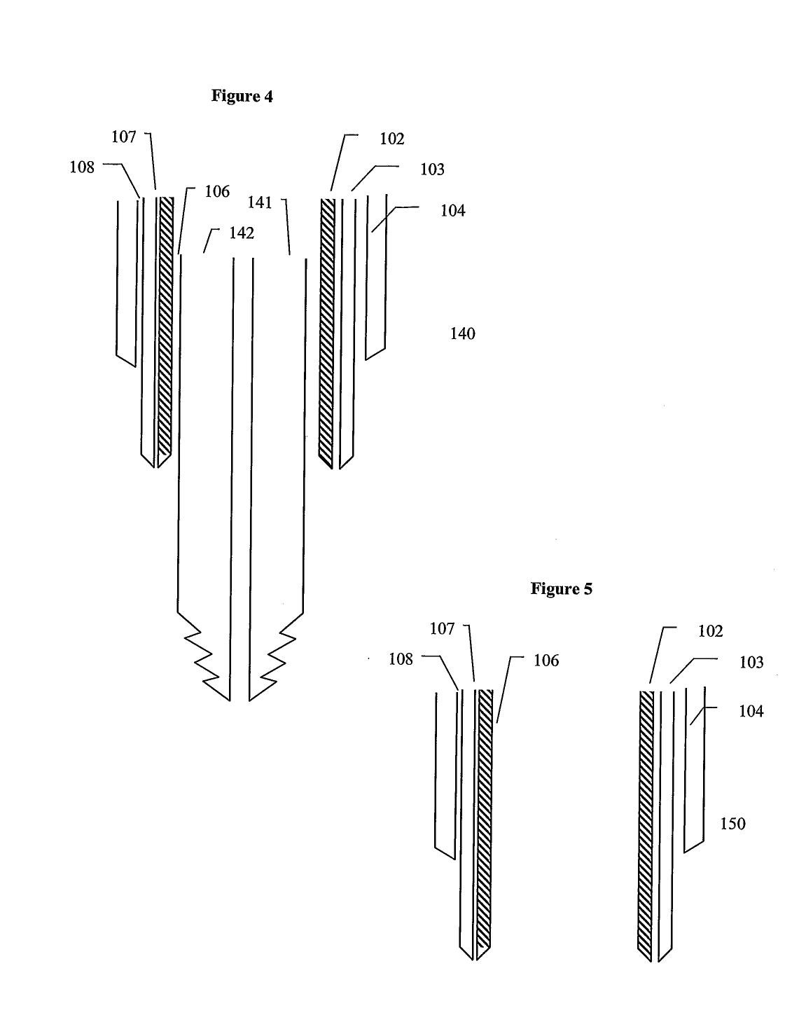

FIG. 4 shows another embodiment of a stylet including two effectors 141 and 142. These effectors have small serrations on the tip, similar to those on the biting parts of a deer fly or the serrations on an electric carving knife or saber saw blades. The preferred motion for effectors 141 and 142 is axial motion, with impulses alternatively being applied to effectors 141 and 142. As one of the effectors is thrust forward, it pushes the other sideways into the tissue, holding the whole needle in place and reducing backsliding of the whole needle assembly. Examples of suitable actuators for this embodiment are also discussed elsewhere herein. The serrations are show as being directed outward in FIG. 4. They could also be directed in a radial direction or be directed inward. The effectors 141 and 142 could be side to side in addition to the edge-to-edge position show. Effectors 141 and 142 could be generally planar or flat, with the serrations being either cutting edges or non-cutting edges. Alternatively, effectors 141 and 142 could be pie shaped segments of a cylinder. In this case the serrations could be non-cutting, and could simply be concentric circles, a spiral pattern, and helical or crossed helical pattern. Other geometrical arrangements of toothed or serrated effectors can be used, with various sizes and depths of serrations.

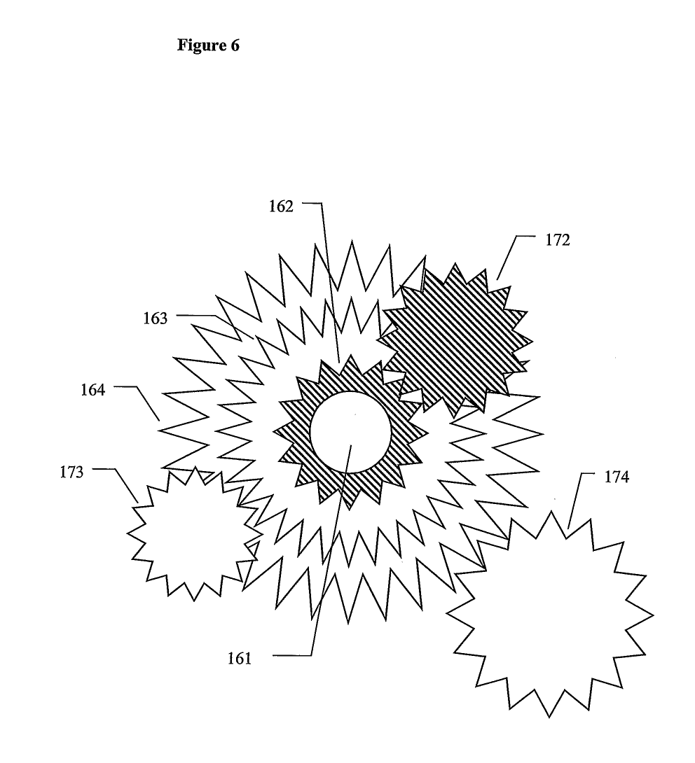

FIG. 5 shows a cross-sectional view of the patient end of an energy assisted needle 150 with the stylet removed. In this configuration, needle 150 is ready to take a tissue sample. In this case, a cutting action is desired because a defined tissue sample is to be removed. In one embodiment, there is relative motion between effector 102 and effector 103. This motion can be continuous rotational motion, intermittent rotational motion, reversing rotational motion, or any of these in combination with axial motion. A cutting action between the edges of effectors 102 and 103 results. The edges of effectors 102 and 103 can be intentionally macroscopically serrated, or they can be ground with a bevel, that on the microscopic level will tend to have serrations as a result of the roughness of the grinding process. In either case these serrations enhance the cutting action. Because the cutting action is a result of the relative motion of the two surfaces, and not a result of the axial force exerted, the benefits of the energy assisted needle described above can be realized.

To allow for or compensate for axial length tolerances, there can be relative axial motion as well as rotational relative motion. The frequency of axial motion can, for example, be an order of magnitude slower than the frequency of rotational motion. Another method of accommodating axial tolerances is to have the bottom edge of effectors 102 and 103 have a macroscopic bevel or wave configuration, so that the relative rotational motion ensures that there is a cutting action over the whole circumference. A further strategy to minimize axial tolerances includes assembling the needle effectors and then grinding the forward ends of the effectors while they are assembled using opposing grinding surfaces (either sequentially or simultaneously) so that a bevel is ground from both sides and meets at the junction of the two effectors.

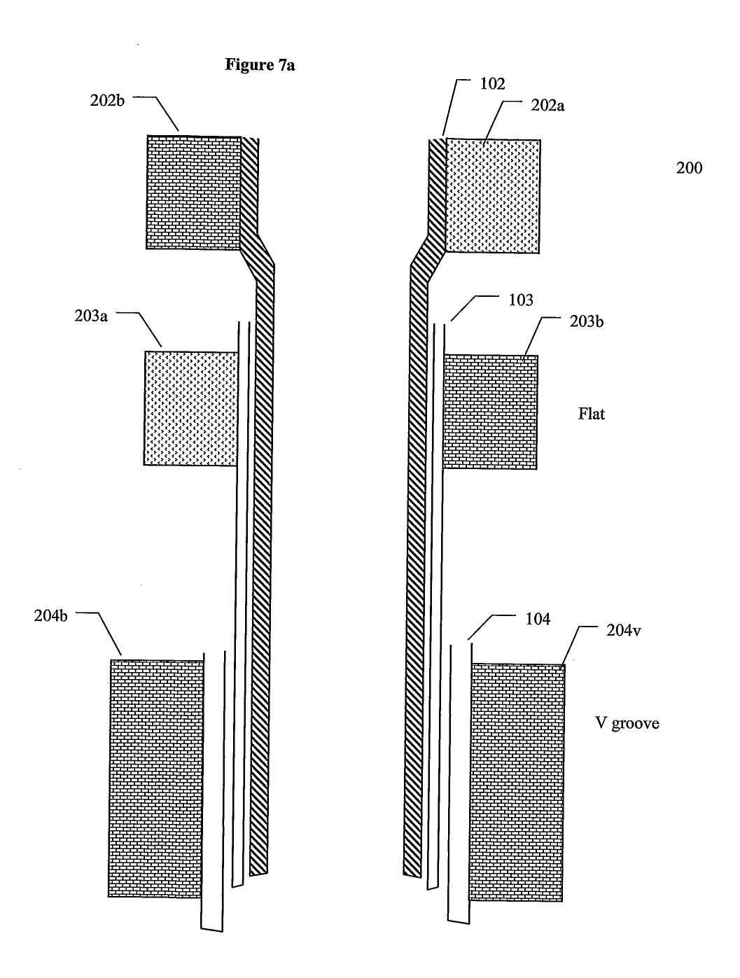

FIG. 6 shows an end on view of the energy assisted needle of FIG. 5. Effector 104 is coupled to gear 164 on the underside or opposite side from this view. Similarly effector 103 is coupled to gear 163 and effector 102 is coupled to gear 162. Hole 161 provides a passage through which a stylet or stylet assembly can be inserted. The stylet (not shown) can also have a gear (not shown) that can be used to couple motion to it. Gear 162 is rotated by gear 172 that is connected to an actuator that can, for example, be an electric motor, rotary solenoid, air motor, or other rotary device. The rotation can be continuous, oscillator, or more complex, as mentioned elsewhere herein. Similarly gear 163 is coupled to gear 173 and thus to a rotary actuator. In one embodiment tube 104 is a sheath that does not rotate, however in some situations such as with a curved needle, it could be beneficial to rotate the sheath for directional control, so gear 164 is shown coupled to gear 174 which can be actuated if beneficial. The motor or rotary actuator can apply continuous, intermittent, oscillator, or arbitrary rotary motion as desired. Other arrangements of gear size and gear placement are possible if needed for packaging optimization. For example, if it is desirable to pull out effectors 102 and 103, for example to remove a tissue sample, the “gear tree” can be constructed with the top gear being the largest gear and the bottom being the smallest.

To allow for axial motion, the planes of meshing gears can be separated by spring elements, for example wave springs, leaf springs, or elastomeric washers. These spring elements allow relative axial motion while rotational motion is occurring. Linear actuators of various types can be used. A rotational - translational arrangement similar to that of U.S. Pat. No. 5,526,882 could be utilized to activate the three elements.

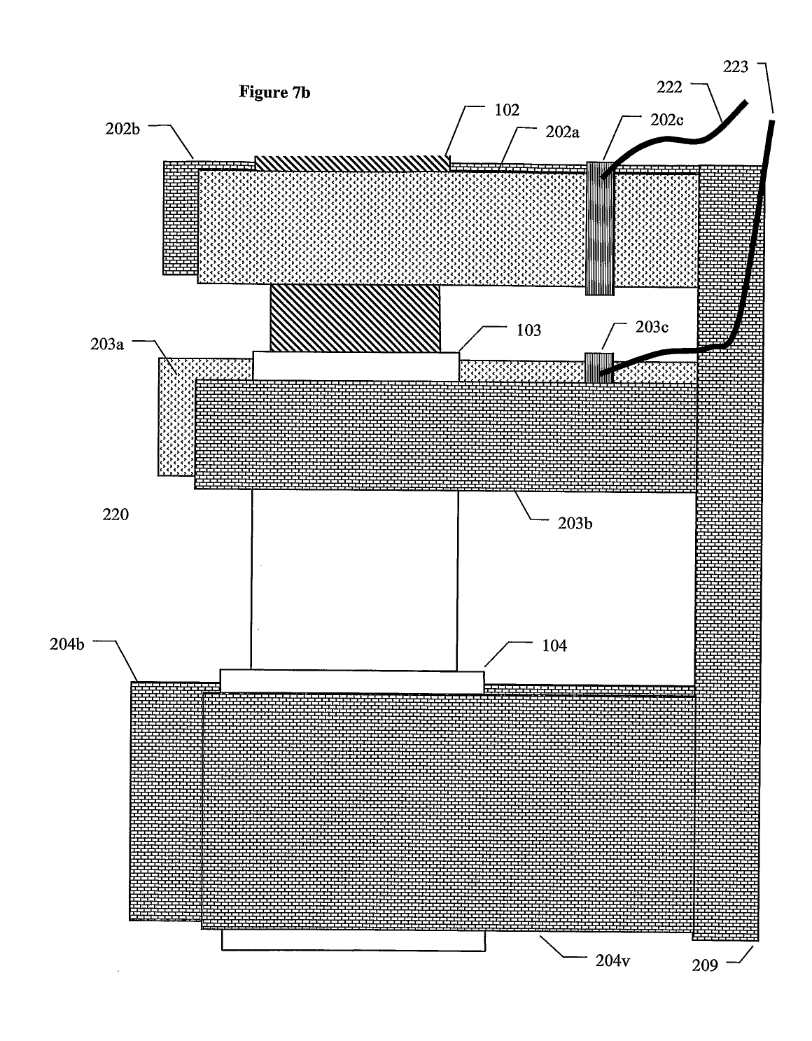

Motors and similar actuators are relatively low speed, although high amplitude actuators. Motors can, for example, operate at 7200 RPM. Some can operate above 10,000 RPM. To get faster motion, especially reciprocal rotary or translational motion, the arrangements of FIG. 7a can be utilized.

In that regard, FIG. 7a is a cross-sectional view and FIG. 7b is a side view of an alternative embodiment for the actuator end of the needle of FIG. 5. The tube that is effector 104 is squeezed between a flat surface 204b and a surface with a vertical V-groove 304v. This V-groove defines a position for the outer effector 104. Effector 103 is gripped between two flat surfaces 203a and 203b of an actuator 203, and effector 102 is gripped between flat surfaces 202a and 202b of an actuator 202. The surfaces 204b, 204v, 203b, and 202b are all rigidly connected together. The surfaces 202a and 203a are moved in an oscillatory in a direction perpendicular to the plane of the diagram. This motion causes elements 103 and 102 to rotate in opposite directions. This motion can, for example, be created by an ultrasonic transducer and horn arrangement with the axis of motion perpendicular to the plane of this drawing. The transducer and horn can, for example, move from 50 to 100 microns at 55 kHz, depending upon the power level supplied. Thus there is 100 to 200 microns of relative motion between the two edges of effectors 102 and 103 in FIG. 5, provided there is no significant attenuation or resonance at that frequency. Resonance can be employed to significantly increase the amplitude of motion. A linear motion of actuator elements 202 and 203 can also be created by other mechanical or electromechanical means, for example air or hydraulic cylinders, solenoids, cams, and electronically excited vibrating springs that act on the actuator arms 202a and 202b and or 203a and 203b in a plane parallel to and distinct from the plane of FIG. 7. The remainder of the elements can, for example, be arranged similarly to that of the elements of ultrasonic scalpels disclosed in U.S. Pat. Nos. 6,514,267 and 6,379,371 that are incorporated herein by reference. It the embodiment described above, there will be a slight bending of elements 102 and 103 as they are moved by 202a and 203a because both 204b and 204v are fixed. This is not a problem if there is sufficient axial distance between 203a and 204a. If there is not sufficient distance, then rather than have 202b and 203b be fixed, they can move in the opposite direct to 202a and 203a (180 degrees out if phase if sinusoidal) so that the elements 102 and 103 experience purely rotational motion and no sideways motion. This can be done by putting actuators in those actuators 202b and 203b and exciting them in opposite phase to that of 202a and 203a.

FIG. 7b shows a side view of the effector actuator assembly of FIG. 7a. The mechanisms that cause linear motion can involve the full actuator 202a and 203a, or can be discrete motive elements that are part of or embedded in actuators 202a and 203a, shown schematically as 202c and 203c, which that are activated through energy supply lines 222 and 223. All actuators are optionally connected to a common frame of reference 209.

FIG. 7c shows a cross sectional view of an alternative embodiment where the actuators are generally parallel to the needle axis. This can provide different packaging and human factors options than the system of FIG. 7a. To create rotary motion, the motion of the actuators is still into and out of the plane of FIG. 7c, but now the actuators themselves move side to side in relation to their length rather than expand and contract in length. This is accomplished by making each motive element 202c and 203c so that it bends instead of simply elongating. This can be done by having two separate elements that elongate next to each other and operated 180 degrees out of phase so that one lengthens when the other shortens, causing the actuators 202a and 203a to bend. To achieve axial motion, all the actuators for an effector are excited in phase, so the effector moves up and down.

In both of these configurations, if the motive elements can cause bending when operated 180 degrees out of phase, they can also cause elongation when operated in phase. And if they are operated out of phase by less than 180 degrees, then both elongation and bending occur. This translates into both rotational and axial motion of the effectors, in this example, the needles. The amplitude and phase can be independently controlled, although the frequency will be the same. The two different actuators can also be driven with different frequencies and amplitudes, so the relative motion can be arbitrarily complex to customize or optimize the cutting action in specific situations.

A benefit of the embodiments shown in FIG. 7a-c is the easy attachment of the effector to the actuator. Depending upon the details of the actuators, the effector can be easily slid and clipped into position from the side or the end, that is to say by moving the effector axially into the actuator, or radially into the actuator with respect to the effector axis. There could be a user control that opens the actuators, or it could simply be that the insertion overcomes the force of a spring that holds the actuators closed. In one embodiment, there is at least one V grove to capture at least one effector. Alternatively, capture could be a simple friction fit, or rely on some other stop or feature.

The effectors can be disposable and new sterile effectors can be used for each patient. It is anticipated that a set of effectors may be used for multiple tissue samples for one patient. In addition, because the energy assist provides cutting with relatively dull edges, it is beneficial when used with cleanable and reusable effectors. Effectors can, for example, be disassembled, cleaned via various liquid solutions know in the art, and then reassembled for safe use with another patient.

While, in one preferred embodiment, both effectors 102 and 103 are moved, it is also possible to move only one of these effectors. For example, if only effector 103 is moved, then the ultrasound energy input to effector 103 could be sufficient that the tissue is cauterized as it is cut. This has the benefit of minimizing bleeding and seeding of any cancerous cells down the needle track as the needle is removed. By not rotating the inner effector 102, the cut tissue sample is collected in effector 102 and is protected from the movement of effector 103. This minimizes the damage to the tissue sample and maximizes its diagnostic value.

The needle can also be operated to switch between the two modes of action described above. The initial penetration or cutting can result from the relative motion of the serrations on the edges of effector 102 and 103. The effector 102 can then be stopped and effector 103 excited with sufficiently increased energy to separate the tissue sample from the remainder of the patient and cauterize the end of the sampling volume.

Alternative methods for separating the tissue core or plug at the end of the sampling include manually provide gross sideways or lateral motion of the needle tip while the cutting energy is still being applied. Alternatively, a corkscrew or spring like element can be inserted in the center lumen to capture and pull out the tissue sample. Furthermore, an energizable wire can be placed across a forward end of the needle tip, and the wire can be energized to separate the tissue. U.S. Pat. No. 6,387,057 disclosed use of a cutting wire on the distal or forward end tissue removing device to assist in separating a tissue core or plug. A device similar to that of U.S. Pat. No. 5,634,473 could be created between effectors 102 and 103 to snare the tissue sample.

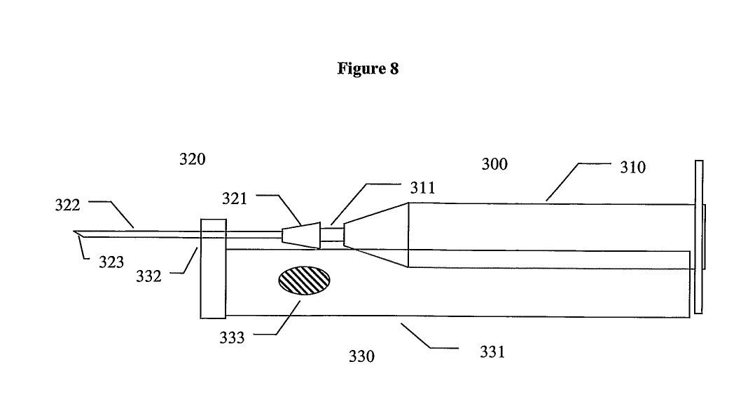

An adaptation to the needle of FIG. 5 to promote ease of tissue cutoff is shown in FIGS. 9a and 9b. The effector 103 is uneven, having a one or more narrow segments 133a that extends axially beyond the end of the cylindrical section 133b. These narrow segments are formed and treated so that their rest position is bent radially inward. When effector 102 in inserted inside 103, the narrow segments are straightened out and rubs against the end 132 of effector 102. This rubbing can provide the close mating to promote good cutting action described above. Moving the whole assembly (effectors 102, 103, and 104) forward while there is relative rotary motion between effectors 102 and 103 will cut a core or plug of tissue. Then to release or separate this core of tissue, effector 103 continues to rotate as it is moved forward, but effectors 102 and 104 stay in place. This allows the one or more segments 133a to bend axially inward as they cut, effectively severing the tissue core from the other tissue and capturing the tissue into the effector 102.

An alternative solution to severing the tissue sample from the body is to have the sample be taken by an effector with the shape of effector 122 in FIG. 3a. In this embodiment, in combination with effector 121, a teasing action is created that moves through tissue without sampling it. To sample the tissue, effector 121 is removed and 122 is energized so that it's macroscopic motion includes coordinated rotation and axial translation. Provided that the edges 122a and 122b are sufficiently sharp to cut tissue, preferably with an energy assistance, a continuous spiral of tissue will be cut and deposited into the core of effector 122. To cut off the core, the axial forward motion is stopped and the effector 122 continues to rotate at least 360 degrees. If the tip of effector 122 comes to the center axis of rotation of the effector, this rotation without translation will sever the tissue. Even if the effector 122 does not come all the way to the axis of rotation, the separation may be sufficient in combination with the curved shape of effector 122 to separate the tissue. The tissue sample can either be removed by removing the needle, or a second sample may be taken at a second location before removal. The samples will simple “stack up” in the effector 122.

There are a number of reciprocating actuators that can provide the linear reciprocation to operate stylet effectors 141 and 142 in FIG. 4. In one embodiment solenoids similar to those used in dot matrix pin printers are used. Examples of such solenoids are described in U.S. Pat. Nos. 4,802,781 and 4,840,501, which are incorporated herein by reference. The solenoid driven pins can be mechanically coupled to the effectors 141 and 142 through friction fitting sleeves, or by other suitably rigid means. Alternatively, the pins of the actuators can end in cups which fit over the ends of effectors 141 and 142 such that only a pushing force can be applied by an actuator pin to effector 141 or 142. The force to hold the effectors 141 and 142 against the actuator pins is provided by the tissue resistance to forward motion. Alternatively, springs can be incorporated to push effectors 141 and 142 against the actuator pins. Alternatively, the actuators could be manufactured as a single piece with the effector. For example, the effector could be partially or totally made from nitinol that changes shape with temperature. The needle of the design of FIG. 4 could be advantageously applied to getting a small blood sample for blood glucose testing. The spacing of effectors 141 and 142 can be selected to optimize blood wicking, so that a sample is drawn into the needle automatically and can then be deposited onto the testing device.

In addition to the flat saw-blade-like effectors 141 and 142, more rounded effectors can be used with the axial motion described above. The effectors can, for example, be pie-shaped in cross section to better fill the tube. There could be more than 2 effectors. The outside of one or more effectors can be serrated or barbed to allow easy forward motion and to resist reverse motion. This leads to the piercing and then teasing apart of the tissue along the path of least resistance.

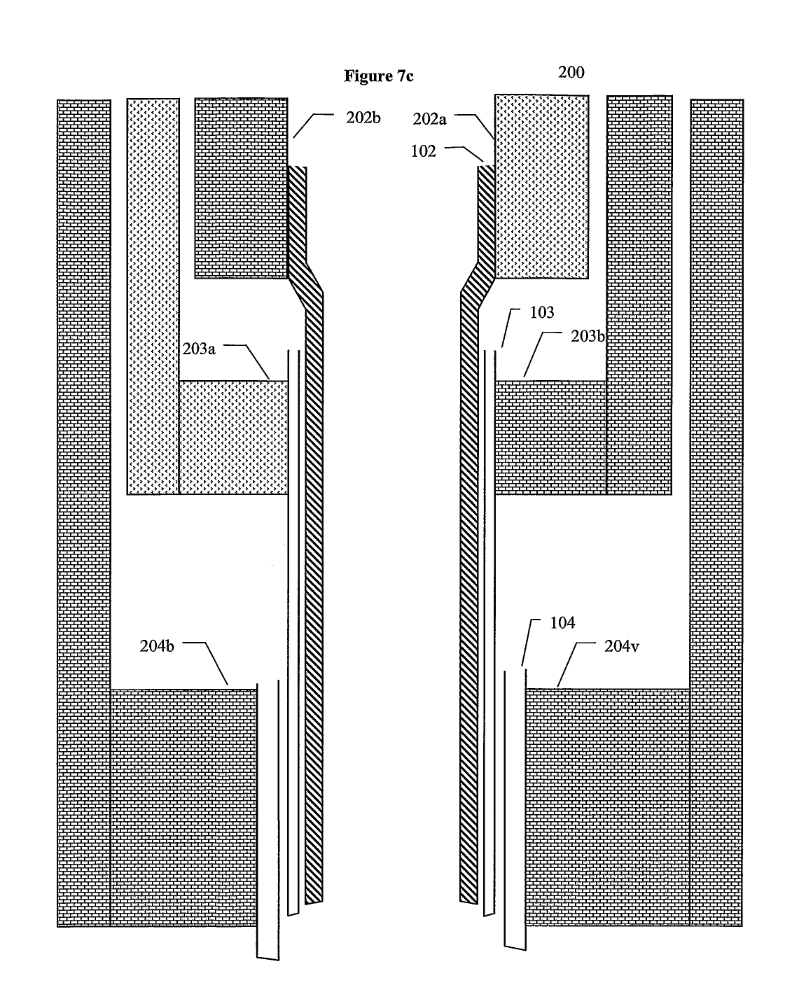

FIG. 8 shows another embodiment of an energy assisted needle 320. In this case, disposable needle 320 has a hollow shaft 322 connected to a hub 321. Hub 321 has a female luer lock that can be tightly attached to syringe 300 by twisting it on to a male luer connection 311. This configuration makes the syringe and needle one relatively rigid body and prevents leakage of fluid from the joint between the needle and the syringe. The fluid in the syringe and the syringe plunger for loading and expelling fluid are not show for simplicity.

By applying an energy assist to needle 320, it can penetrate the skin more easily and thus the forward thrust force is reduced (or even eliminated). This energy assistance allows a smaller diameter needle to be used, reducing the pain and tissue damage. Needles of the present invention can, for example, have a diameter of 0.25 inches or less. Indeed, needles of the present invention can have a diameter of 0.1 inches, 0.01 inches or less. This is of great benefit, for example, to patients who require frequent and long-term injections of medications, such as insulin dependent diabetics.

Syringe 300 and attached needle 320 are mounted in an energizer 330. The energizer 330 includes an actuator 332 that grips shaft 322 of needle 320. The gripping connection can for example be a friction grip similar to that discussed in connection with FIG. 7a-c or other arrangements know to those skilled in the art.

Actuator 332 can, for example, be a piezoelectric stack that operates as described in connection with FIGS. 7a-c. The user interface to power controller 51 in this case is a button 333. When the user activates/pushes button 333, an internal switch is closed. In this case, the switch is power controller 12 that allows power to go from power source 11 (for example, a battery) to a drive circuit, both or which can be contained in housing 331, which energizes the piezoelectric elements in actuator 332.

In an alternative embodiment, needle shaft 322 can have an adapter attached to it to facilitate the coupling to actuator 332. For example, concentric gears can be provided as described in connection with FIG. 6. In that case, actuator 332 can be a mating gear connected to a motor in housing 331, which is energized by switch 333.

In one embodiment, actuator 332 provides rotational motion to the needle shaft 322. Actuator 332 can also provide axial motion or both rotational and axial motion. Preferably, lateral motion is sufficiently small to prevent needle shaft 322 from buckling as it is being inserted into the patient.

In one embodiment, actuator 332 preferably mounted ¼ of a wavelength from the hub 321 at the frequency used. Needle tip 323 can be positioned n/2 wavelengths from the actuator 332. This configuration assists in ensuring that the movement at hub 321 is minimized and the movement at the needle tip is maximized. The wavelength is a function of needle shaft 322 material properties and dimensions. If it not convenient or desirable to have this spacing, then instead of the rigid adhesive connection between shaft 322 and hub 321, a thicker section of a more flexible adhesive, such as silicone could be employed. Such a flexible adhesive or other coupling accommodates the rotation (and/or other motion) of needle shaft 322 without causing significant rotation of hub 321.

In an alternative embodiment, actuator 332 energizes both needle 320 and syringe 300. Because the mass being energized is significantly higher, it is likely that lower frequency motions will be desirable. This embodiment has the benefit of allowing commonly available syringes and needles to be used. However, there still can be a benefit to having a custom locking shape. For example, the hub can have gear teeth on the outer surface, to match with a gear in the actuator. Or, the syringe luer or neck 311 could have flat elements to better mate with flat elements on the actuator and provide more positive energy transfer.

For simplicity, needle shaft 322 may be a single effector. Alternatively, the needle shaft may utilize several effectors in any of the arrangements discussed above.

Intravenous catheters, normally the catheter over needle type, serve as tissue resident conduits for administering or removing material. They are often used instead of intravenous needles for the injection of drugs because a sharp needle left in a vein can easily penetrate outside the wall of the vein if the patient moves his or her limb, even if the needle hub is taped to the patient's skin. Sharp, rigid metal needles are commonly used for delivering medicine or drawing blood by hand, when the operation is all done at one time and the needle is supported by the doctor, nurse, or operator. In situations such as CT contrast injections, there is usually a time of 5-10 minutes to an hour or more between insertion of the catheter and the injection of the fluid. During that time the patient will be able to move the limb with the catheter. During IV fluid administration the duration of fluid administration is many minutes to hours. Catheters are commonly used as fluid conduit to other tissue as well. The same distinction exists in this case, rigid metal needles are generally held by the operator or a fixture during the procedure, whereas catheters are stabilized on or in the patient and the patient is relatively free to move, with restrictions based upon the specifics of the situation. However, because an energy assisted needle can be a relatively poor cutter when there is no energy applied and relatively good cutters only when energy is applied, and the cutting action is relatively decoupled from the forward thrust down the length of the needle, energy assisted needles made from metal, relatively rigid plastics, or flexible plastics could replace catheters in many applications. This has the benefit that for a given outside diameter and pressure capability, an energy assisted needle can have a larger inside diameter than a soft plastic catheter.

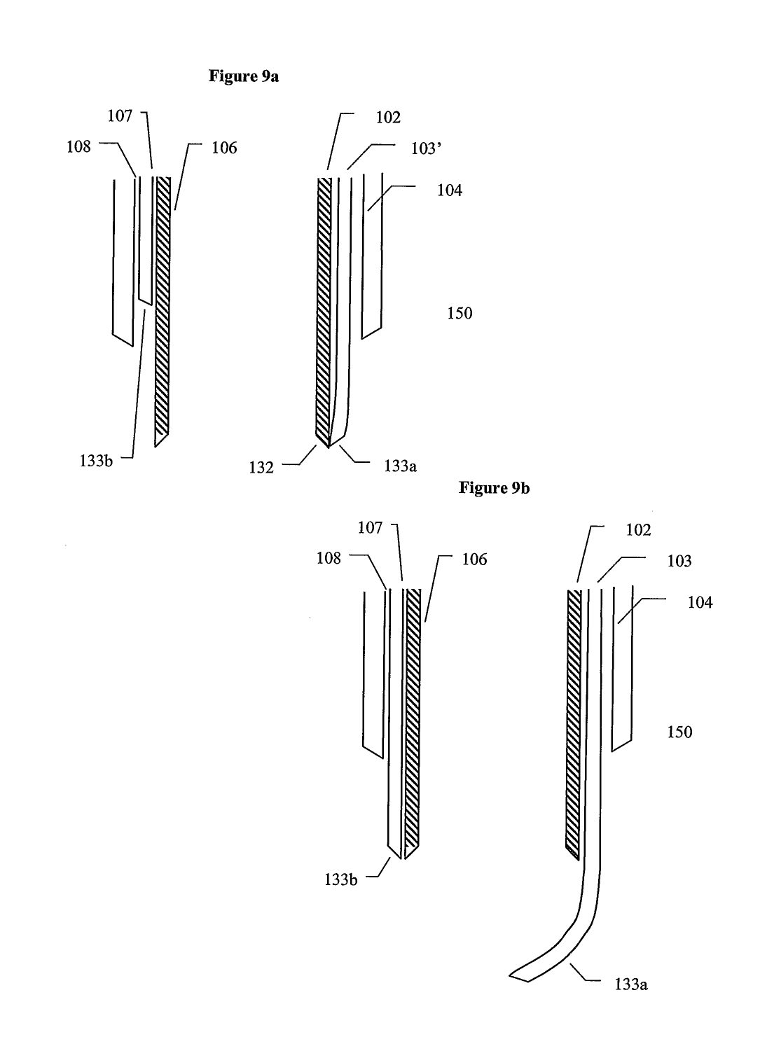

Alternatively, the needle in the normal catheter over needle design could be given an energy assist to make penetration of the vein easier and eliminate the problem of the vein moving out of the way. FIG. 10a shows a detail of the catheter over needle tip and FIG. 10b shows the catheter needle assembly 400 mated with an actuator, power source, power controller, and user interface. The energy assisted penetration action comes from the relative motion of effectors 121 and 122, as was discussed in relation to FIGS. 3a and 3b. In this embodiment, instead of effectors 102, 103, and 104, a relatively flexible effector 401 encloses and is preferably frictionally associated with effector 122. The catheter has a luer connector 421 that is attached to the flexible effector 401 and is subsequently used to connect to fluid lines or a syringe. The effectors 121 and 122 are energized, the overlying tissue is traversed, the blood vessel wall is penetrated, and then catheter 400 is slid forward into the vessel and effectors 121 and 122 are removed from the catheter and disposed of.

The hand held energizer 330, similar to that in FIG. 8, includes an actuator 332 that grips effectors 121 and 122. The gripping connection can for example be a friction grip similar to that discussed in connection with FIGS. 7a-c, the gear arrangement of FIG. 6, or other arrangements know to those skilled in the art. The user holds the case or housing 331 and selectively activates the energy assist through switch 333. They guide the needle into the blood vessel either visually or with the assistance of some guidance system. Once in the vessel, the effectors 121 and 122 are separated from the hand held energizer 330 and disposed of. The hand held energizer 330 can be reused, although it could also be disposable if it were inexpensive enough, for example a spring driven assembly. If it is reusable, the hand held energizer 330 should be cleanable, preferable with liquid cleaners. In addition it is preferable that the details of the mating arrangement with effectors 121 and 122 are such that the hand held energizer 330 does not contact the luer connector 412 of the catheter 400 to preserve the sterility of the luer connector. A simple way to achieve this is to have a cap on the luer 421 that is also disposed of. This cap could include a flexible septum so that blood does not flow out the catheter when effectors 121 and 122 are removed.

To simplify the hand held energizer 330, rather than having independent actuators for effectors 121 and 122, effectors 121 and 122 could be mechanically coupled to each other so that motion of one produced a delayed motion in the other. This could be as simple as a spring and mass relationship. If this relationship has a resonance, and it is excited by a reciprocating motion near that resonant frequency, then the motions of effector 121 and 122 can be 180 degrees out of phase. Thus with just one actuator, the augmented penetration can be accomplished. In the case where two effectors relate to each other through a spring or other elastic or deformable member, the second effectors can be short, meaning that it does not have to run the full length of the needle and separately attach to an actuator. The second effector can interact with the first effector anywhere along the length of the needle. This has the benefit of decreased mass of the second effector, higher resonant or response frequency and simplifying the construction. Of course the second effector could run the fall length with the spring connection being at the proximal end. This could have the benefit of increasing mass, lower resonant or response frequency, and increased structural rigidity.

A further simplification can occur by eliminating one of the effectors, for example effector 121. If effector 122 is excited at a frequency sufficient that tissue cannot move out of it's way quickly enough, it will cut or tease its way through the tissue. Effector 401 further acts as a dilator, widening the opening in the vessel wall as it penetrates.

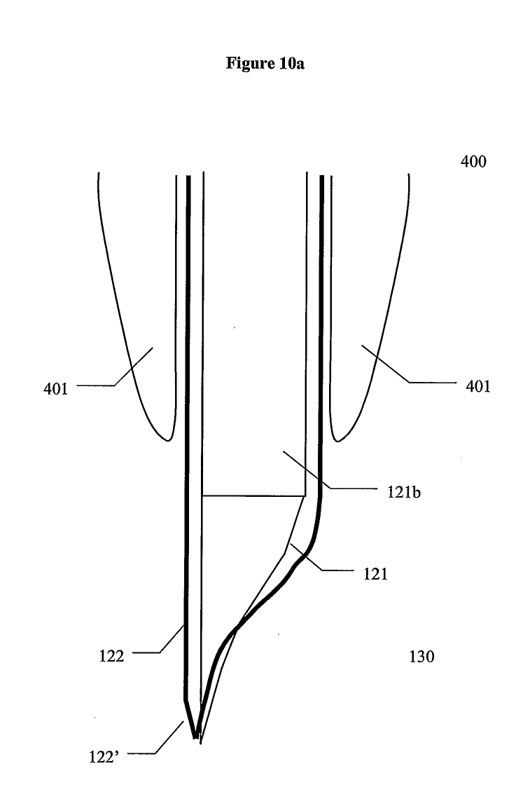

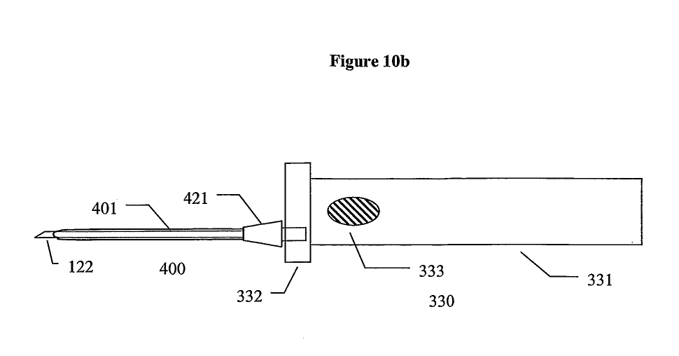

FIGS. 11d, 11e & 11f show a modified effector tip design 460 in a side, front, and back view respectively, including a “W” or multi-tip design that facilitates the capturing and piercing of a blood vessel for entry by a needle or catheter. The simplest way to understand this tip is to consider a current non-coring needle 450 show in FIGS. 11a, 11b, & 11c in a side, front, and back view respectively. There is a single point 451 that penetrates the tissue when the operator pushes it. Edges 453 are cutting edges. Edge 455 is a non-cutting edge that simply moves the tissue out of the path. This is what prevents the cutting of a core.

In effector tip design 460 the spaced, dual (or more) tips 461 and 462 of effector 460 are created by grind off the tip 451 of a non-coring needle at an angle creating edge 467. The angle of edge 467 is chosen so that at the normal “angle of approach” to the vessel, the tips 461 and 462 contact the vessel rather than point 469. The normal angle used is 10 to 20 degrees, somewhat determined by the tendency of the vessel to move or roll when force is applied to puncture it and to avoid puncturing out the other side of the vessel because of the “jump” that comes from breaking through the vessel wall. Both or these problems are at least partially mitigated by an energy assist. The two points 461 and 462 with a middle groove 463 facilitate centering of the effector on the vessel before cutting into it. Two concentric effectors conceptually similar to those of FIG. 3c could provide for energy assisted teasing or tearing action at the two points 461 and 462. Or a single effector can be moved rapidly enough that the cutting action occurs without the need for the second effector. This tip design can be advantageously employed without an energy assist as well. In this case edges 464 and 467 are cutting edges as in the prior art needle, and edge 465 is a non-cutting edge. Even with the energy assist, it could be advantageous to turn off the energy assist momentarily to allow centering the effector on the vessel, and then turn the energy assist back on for piercing. This and similar tip designs can also be advantageously applied in any situation in which a tougher tissue, for example a tumor, is being targeted for piercing.

A third option is to use the energy assisted needle to improve the needle over catheter design. The normal needle over catheter has a catheter inside a needle, and after penetrating the vein wall, the catheter is pushed forward into the vein and the needle is withdrawn back the shaft of the catheter. The needle is then split from around the catheter along a thinned lateral line. The device would be similar to that of FIG. 10a, with the exception that the actuator grips or interacts with the needle on the distal side of the luer connection. Again, the sterility of the luer needs to be preserved. Currently needle over catheter designs have fallen out of fashion except in selected applications because of the difficulty in removing the needle. Energy assisted cutting can be advantageously used with currently available needle over catheter designs. In addition, if a single or dual effector energy assisted needle were used, the needle need not be a fall cylinder, but could just encompass the catheter for somewhat more than half a circle, more than 180 degrees. The assembly could be similar to that of FIG. 3 where effector 121 is the plastic catheter, effector 122 is metal, and the other effectors are absent. To insert the IV catheter, a device similar to that of FIG. 8 or 10a-b energizes the needle. After the vessel wall is pierced, the needle is slid back and the catheter is simply pulled from the needle. There is no need to split the needle. The flexibility in the plastic enables it to be pulled from the needle's grip. This embodiment has the benefit that the catheter does not have to have an end hole. It can, for example have many side holes or slits to disperse the fluid being injected and to avoid a jetting effect that can damage the vessel wall.

In IV catheter embodiments of the present invention, the forward end of the effector or the effector tip can, for example, be similar to that of effectors 102 and 103. The effector tip can have a macroscopic bevel as current needles. In certain embodiment, in can be preferable that the energy assisted cutting action take place only in a region +/− approximately 45 degrees to +/− approximately 90 degrees from the beveled tip. This region of cutting action facilitates the location of the cutting region of the needle against the center of the vein to be penetrated and reduces the chance of coring.

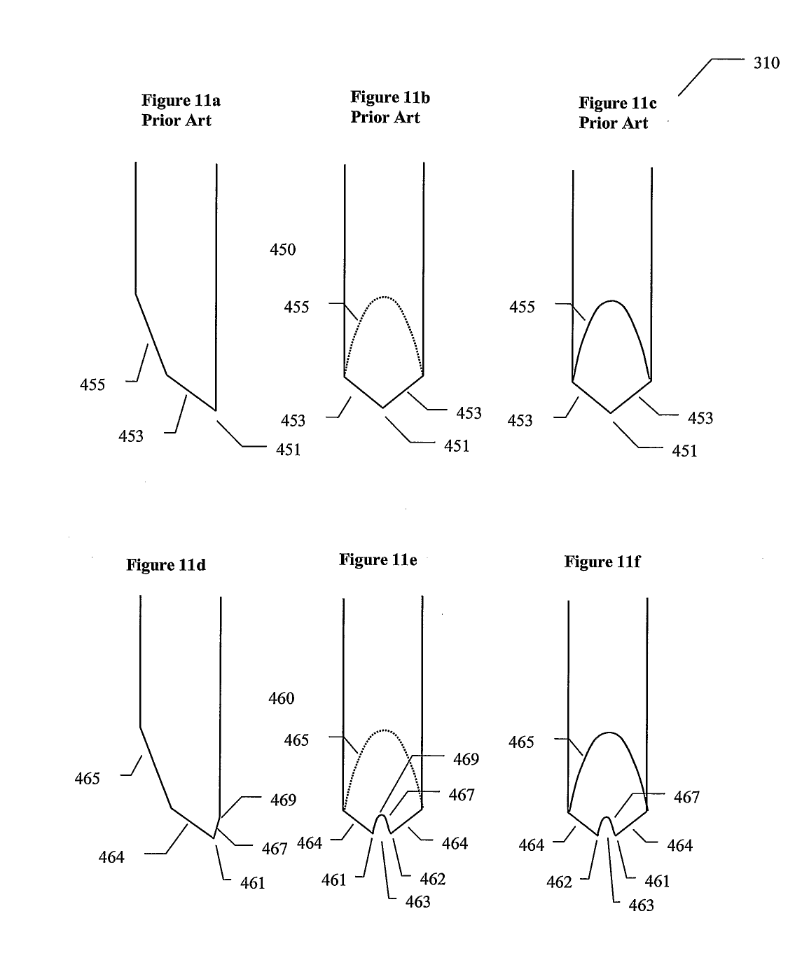

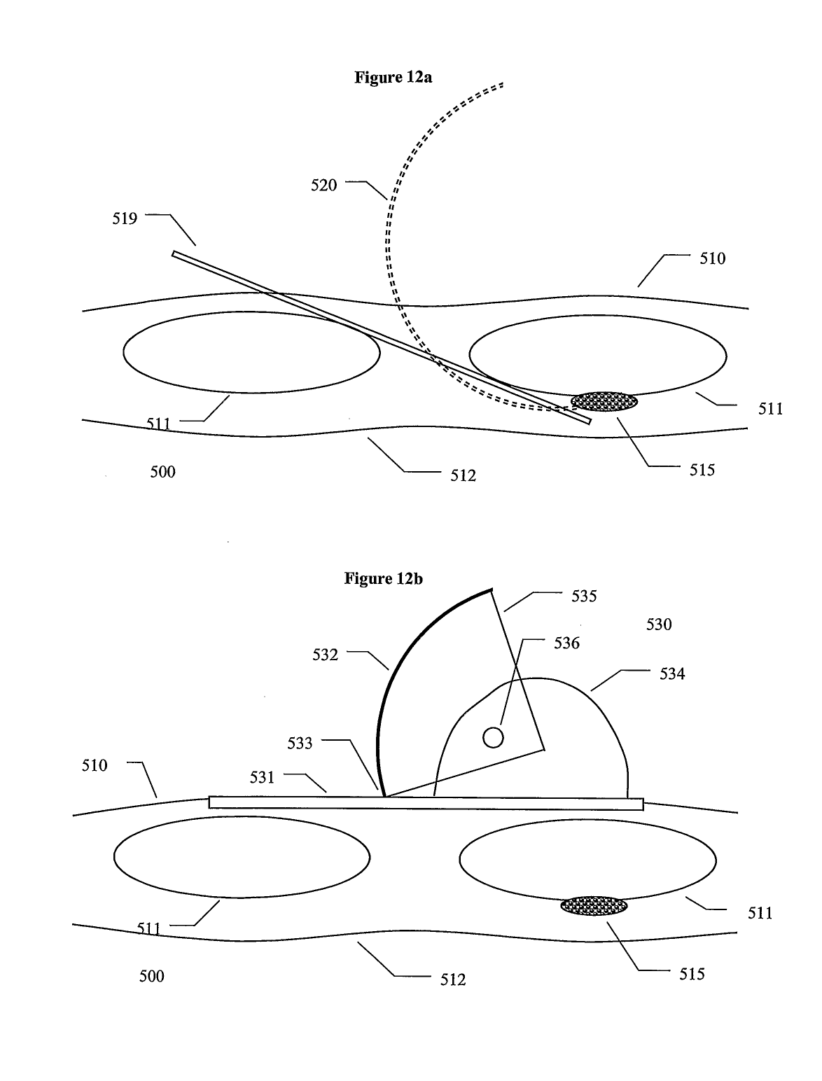

FIG. 12a shows a cross section of a patient's anatomy 500, illustrating a situation in which an energy assisted curved needle and guide is advantageous. The skin surface is 510. There are two ribs 511, and the pleural space begins at surface 512. To biopsy a suspected lesion 511 that is under and close to a rib 511, a straight needle path 519 cannot be used, but a curved needle path 520 could be used. 510. It is desirable to not traverse or transect the pleural space.

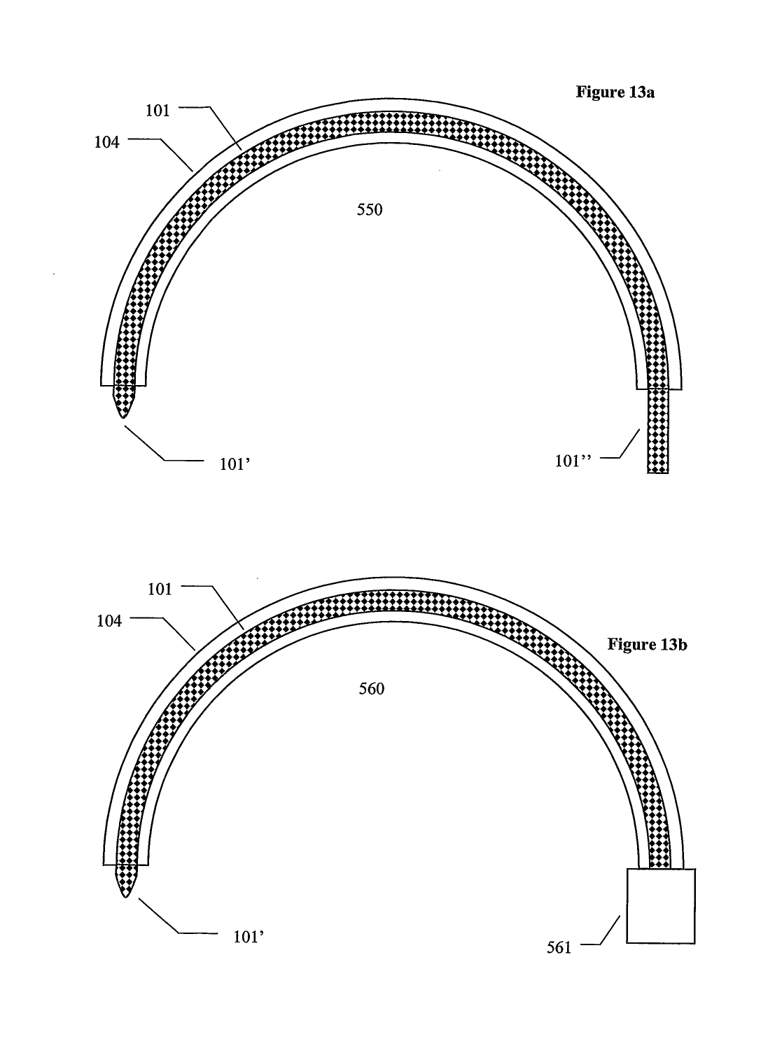

FIG. 13 shows a curved needle 550. This is the simplest type, with just two effectors 101 and 104. Effector 104 is a sufficiently rigid, hollow tube. Effector 104 defines the curve or shape of the device. Effector 101 is a torqueable effector sized and made from lubricious materials so that it can be moved within effector 104. It can be constructed using various arts, for example those of building flexible shafts or torqueable guidewires and catheters. The flexible shaft art includes braided or wound wire flex shafts, tightly coiled springs, and flexible wire inside a housing. The catheter and guidewire art enables several concentric flexible effectors to be built, installed and operated within a relatively small diameter shape defining effector 104. The patient or distal end of the effector 101 indicated as 101′ illustrates the simplest option, that similar to FIG. 2 where the shape is a simple point, similar to that in FIG. 2. Using the catheter and guidewire design and manufacturing arts, effectors similar to those of FIG. 2, 3, 4, 5, 9, 10, or 11 could operate in a curved path. The curved needle is show with an arc of 180 degrees for clarity of understanding. The arc could be as little as 60 degrees, or even less. The preferable arc length is between 60 and 135 degrees. An angle approaching 180 degrees would make it difficult to start the needle into the skin without also hitting the skin at the other end.

The curve of needle 550 is all in the plane of the paper in FIG. 13. It could optionally curve in a complex manner, for example it could be a spiral, a spiral with a curved enter axis, or an arbitrary curve that does close on itself. A spiral has the advantage of overcoming the limitation mentioned above when going above about 135 degrees of arc. A spiral would enable more than 360 degrees of arc to be used, because the needle can spiral up away from the skin.

The curved needle of FIG. 13b is steerable needle 560. It includes a steering mechanism 561, that for example could be electronic including thermobending element at the tip or along the length, or that is mechanical, using cables as has been done in endoscope or laparoscope design as in for example U.S. Pat. No. 6,458,076 which included herein by reference. The user controls the penetration direction and thereby the path of penetration through a user interface that is either part of the steering mechanism 561 or in communication with the steering mechanism 561.

One of the challenges in the use of a curved needle is guiding it, since the current training and experience is with straight needles. The use of a curved needle guide 530 is illustrated in FIG. 12b. The needle guide 530 consists of a movable element 535 with a guiding surface 532 matched to the curvature of the needle, grooved or otherwise constructed to minimize undesired lateral motion while allowing motion along the curve. The movable element 535 is attached to a mounting base 531. Mounting base 531 can, for example, be attached adhesively to the patient's skin. It will tend to planarize or flatten the skin in this area. There needs to be an opening, not shown for the needle to go through the base and into the patient. If the guide 530 is plastic and thereby preferable disposable, one option for attachment of the movable element 535 is to have a living hinge at the point 533. To hold the guide at the proper angle, support 534 is rigidly attached to the base 531. An activatable attachment element 536 fixes and holds the relative position between movable element 535 and support 534. The activatable attachment can be, for example, adhesive, Velcro, a screw and wing nut, or spring biased ratchets. The guide can be operated manually, or can be used in conjunction with an imaging system to allow the operator to position the movable element at the proper position. To avoid having to pull the guide off the patient, the base 531 could be of two segments, that allow synchronous lateral translation of the movable element 535 and the support element 534. The guide would be especially useful with a 3D guidance system.

The curved needle can be used for all the uses discussed herein, for example to sample tissue, that is to take a biopsy, to place stitches, or remove or inject fluids. For longer-term fluid delivery or sampling, the curved needle can be utilized with the catheter structure discussed in respect to FIG. 10a. The curved needle allows a catheter to placed through tissue in a curved path, which could have many advantages in patient care. In the case of placing stitches, the needle could be solid and one piece. The energy assist could be provided by having forceps that include actuators that supply the energy to the needle. Likewise, a hollow needle could have a single effector, and some benefits of energy assisted piercing and cutting would still be realized. Optionally, a second effector need not run the length of the needle. It could be attached to the first effector through an elastic member such that there is relative motion between it and the first member as mentioned elsewhere.

Although the present invention has been described in detail in connection with the above embodiments and/or examples, it should be understood that such detail is illustrative and not restrictive, and that those skilled in the art can make variations without departing from the invention. The scope of the invention is indicated by the following claims rather than by the foregoing description. All changes and variations that come within the meaning and range of equivalency of the claims are to be embraced within their scope.

What is claimed is:

1. A device for penetrating tissue and removing a biological sample, comprising:
a biological sampling element to remove the biological sample, the biological sampling element including a passage therethrough; and
a penetrator positioned within the passage, the penetrator being energized in a repetitive manner to assist in penetrating tissue.

2. The device of claim 1 wherein the penetrator is energized continuously to assist in penetrating tissue.

3. The device of claim 1 wherein the penetrator is energized for discrete periods of time.

4. The device of claim 1 wherein the penetrator is energized in a manner to cause motion of the penetrator.

5. The device of claim 1 wherein the penetrator is energized to cause heating of the penetrator.

6. The device of claim 4 wherein the motion of the penetrator includes at least one of rotational motion or axial motion.

7. The device of claim 4 wherein the penetrator includes at least a single effector that is moved.

8. The device of claim 4 wherein the penetrator includes a plurality of effectors, at least one of which is moved.

9. The device of claim 4 wherein the penetrator comprises at least two effectors in close proximity to each other, relative motion between the two effectors assisting penetration of tissue via interaction with tissue in regions where there is close proximity of tissue to an interface between the two effectors.

10. The device of claim 4 wherein the penetrator includes at least two effectors, including a first effector which is moved and a second effector in proximity to the first effector which is stationary, the first effector and the second effector cooperating to penetrated tissue via interaction with tissue in regions where there is close proximity of tissue to an interface between the first effector and the second effector.

11. The device of claim 4 wherein the penetrator includes at least two effectors, including a first effector which is moved and a second effector in proximity to the first effector which is also moved, the first effector and the second effector cooperating to penetrate tissue via interaction with tissue in regions where there is close proximity of tissue to an interface between the first effector and the second effector.

12. The device of claim 1 wherein the biological sampling element comprises:
a first tubular structure
a vibrational coupler that couples rotational energy into the first tubular structure, such that the vibrational energy cuts tissue at the leading edge of the first tubular structure;
a second tubular structure inside said first tubular structure such that the cut tissue inside the second tubular structure is protected from the effect of the rotational energy of the first tubular structure, the penetrator passing through the second tubular structure.

13. The device of claim 1 wherein the biological sampling element is adapted to remove a tissue sample.

14. The device of claim 13 wherein the biological sampling element is adapted to cut tissue and remove the tissue sample.

15. The device of claim 1 where in biological sampling element is adapted to remove a sample of biological fluid.

16. The device of claim 15 wherein the biological fluid is blood.

17. The device of claim 1 wherein electrical energy is used in energizing the penetrator.

18. A device for penetrating tissue and positioning a catheter, comprising:
a catheter comprising a passage therethrough; and
a penetrator in operative connection with the catheter, the penetrator being energized in a repetitive manner to assist in penetrating tissue.

19. The device of claim 18 wherein the penetrator is removably positioned within the passage of the catheter.

20. The device of claim 18 wherein the penetrator is positioned on the exterior of the catheter.

21. A needle for penetrating tissue comprising:
a first effector comprising a surface; and
at least one actuator in operative connection with the first effector, the actuator adapted to cause motion of the first effector such that tearing of tissue takes place in regions where there is close proximity of tissue to the surface of the first effector.

22. The needle of claim 21 wherein the surface of the first effector is a forward surface thereof.

23. The needle of claim 23 wherein the forward surface of the first effector is rough.

24. The needle of claim 21 wherein the needle penetrates without application of a significant axial force thereto.

25. The needle of claim 21 wherein tissue is torn along a path determined by the characteristics of the tissue.

26. The needle of claim 25 wherein the path is determined at least in part by the resistance to tearing exhibited by tissue forward of the needle.

27. The needle of claim 25 wherein tissue having a relatively higher resistance to tearing is pushed aside by the needle and not torn.

28. The needle of claim 21 further comprising at least a second effector comprising a surface, the surface of the second effector being in close proximity to the surface of the first effector; relative motion between the first effector and the second effectors causing tissue tearing to occur in regions where there is close proximity of tissue to an interface between the first effector and the second effector.

29. A needle for sampling tissue, comprising
a first tubular structure;
a vibrational coupler that couples rotational energy into the first tubular structure, the vibrational energy being suitable to penetrate tissue at the leading edge of the first tubular structure;
a second tubular structure positioned inside the first tubular structure, such that cut tissue passes into the second tubular structure and is protected from the effect of the rotational energy of the first tubular structure.

30. A method of inserting a tissue resident conduit into tissue, comprising the step:
energizing at least a portion of a forward end of the a conduit insertion device to assist in penetrating tissue.

31. The method of claim 30 wherein the tissue resident conduit is a catheter.

32. The method of claim 30 wherein the tissue resident conduit is flexible.

33. The method of claim 30 wherein the tissue resident conduit has a blunt forward surface.

34. A device for inserting a tissue resident conduit comprising:
at least one component that is energized during penetration to assist in penetrating tissue.

35. The device of claim 34 wherein the tissue resident conduit is flexible and the energized component is positioned on a forward end of the tissue resident conduit.

36. The device of claim 35 further comprising a mechanism for directing the penetration of the tissue resident conduit.

37. The device of claim 34 further comprising a rigid penetrator, the energized component being positioned on a forward end of the penetrator, the tissue resident conduit being in operative and removable connection with the penetrator so that the penetrator can be removed from penetrated tissue while the tissue resident conduit remains within the penetrated tissue.

38. The device of claim 37 wherein the penetrator comprises an axial passage therethrough in which the tissue resident conduit is positioned during penetration.

39. The device of claim 37 wherein the penetrator is positioned within the conduit during penetration.

40. The device of claim 37 wherein the tissue resident conduit is positioned adjacent the penetrator during penetration.

41. The device of claim 37 wherein the tissue resident conduit is flexible.

42. The device of claim 34 wherein the tissue resident conduit is a catheter.

43. The device of claim 37 wherein the tissue resident conduit is a catheter.

44. The device of claim 34 wherein the effector is adapted to penetrate through a wall of a blood vessel.

45. A device for penetrating tissue comprising:
a nonlinear penetrator comprising at a forward end thereof at least a first effector, the device further comprising at least one actuator in operative connection with the first effector, the actuator adapted to cause motion of the first effector.

46. The device of claim 45 wherein the penetrator is curved with a curve of a predetermined shape.

47. The device of claim 46 wherein the penetrator is curved in a complex manner.

48. The device of claim 45 wherein the penetrator is flexible.

49. The device of claim 45 further comprising a mechanism to direct the penetration of the penetrator.

50. A device for penetrating tissue comprising:
a penetrator comprising at a forward end thereof at least a first effector and at least one actuator in operative connection with the first effector, the actuator adapted to cause motion of the first effector, the effector being rotatable about the axis of the penetrator

51. A non-coring needle comprising a penetrating member, a forward end of the penetrating member comprising a forward extending section comprising at least two points spaced from each other and being adapted to pierce tissue.

52. The needle of claim 51 further comprising an actuator to energize at least a portion of the needle to facilitate penetration.

53. The needle of claim 51 wherein at least a portion of the forward end of the penetrating member is non-cutting so that coring does not occur upon penetration of the tissue.

54. The needle of claim 51 wherein the at least two point are positioned to stabilize tissue for penetration.

55. A blunt needle comprising at least one effector that does not readily penetrate tissue and at least one actuator that when energized enables the needle to readily penetrate tissue.

56. A needle of claim 55 containing a conduit such that fluid can be delivered to the tissue or material removed from the tissue.
